SEQUENCE LISTING (29 sequences)

SEQ ID NO: 1 - RNA, 19 nt, Human immunodeficiency virus
acaggagcagaugauacag

SEQ ID NO: 2 - RNA, 19 nt, Human immunodeficiency virus
auggcaggaagaagcggag

SEQ ID NO: 3 - RNA, 19 nt, Human immunodeficiency virus
gaagaaaugaugacagcau

SEQ ID NO: 4 - RNA, 19 nt, Human immunodeficiency virus
gagagagaugggugcgaga

SEQ ID NO: 5 - RNA, 19 nt, Human immunodeficiency virus
gugaaggggcaguaguaau

SEQ ID NO: 6 - DNA, 34 bp, Artificial
  primer
cacacacaaggctacttccctgattagcagaact

SEQ ID NO: 7 - DNA, 41 bp, Artificial
  primer
accttgctgcagcctctatcttgtctaaagcttccttggtg

SEQ ID NO: 8 - DNA, 31 bp, Artificial
  primer
gaagcagaagttatcccagcagagacagggc

SEQ ID NO: 9 - DNA, 34 bp, Artificial
  primer
cccaagcttctaatcctcatcctgtctacttgcc

SEQ ID NO: 10 - DNA, 44 bp, Artificial
  primer
accttgtctagaatggagccagtagatcctagactagagccctg

SEQ ID NO: 11 - DNA, 31 bp, Artificial; identical to SEQ ID NO: 8
  primer

SEQ ID NO: 12 - RNA, 19 nt, Human immunodeficiency virus
aggagagagaugggugcga

SEQ ID NO: 13 - RNA, 19 nt, Human immunodeficiency virus
ggagagagaugggugcgag

SEQ ID NO: 14 - RNA, 19 nt, Human immunodeficiency virus
agagagaugggugcgagag

SEQ ID NO: 15 - RNA, 19 nt, Human immunodeficiency virus
gagaugggugcgagagcgu

SEQ ID NO: 16 - RNA, 19 nt, Human immunodeficiency virus
aaggugaaggggcaguagu

SEQ ID NO: 17 - RNA, 19 nt, Human immunodeficiency virus
aggugaaggggcaguagua

SEQ ID NO: 18 - RNA, 11 nt, Human immunodeficiency virus
ugugcccgugu

SEQ ID NO: 19 - RNA, 11 nt, Human immunodeficiency virus
agaugggugcg

SEQ ID NO: 20 - RNA, 26 nt, Human immunodeficiency virus
aggagagagaugggugcgagagcguc

SEQ ID NO: 21 - RNA, 26 nt, Human immunodeficiency virus
aggagagaaaugggugcgagagcguc

SEQ ID NO: 22 - RNA, 26 nt, Human immunodeficiency virus
aggagagagauggguacgagagcguc

SEQ ID NO: 23 - RNA, 26 nt, Human immunodeficiency virus
aggagagagaugggcgcgagagcguc

SEQ ID NO: 24 - RNA, 26 nt, Human immunodeficiency virus
aggagagagaugggugugagagcguc

SEQ ID NO: 25 - RNA, 11 nt, Human immunodeficiency virus
ugugcccgucu

SEQ ID NO: 26 - RNA, 49 nt, Artificial
  RNA in expression cassette
gugccuggcuagaagcacauucaagagaugugcuucuagccaggcacuu

SEQ ID NO: 27 - RNA, 21 nt, Artificial
  after DICER
gugccuggcuagaagcacauu

SEQ ID NO: 28 - RNA, 21 nt, Artificial
  after DICER
ugugcuucuagccaggcacuu

SEQ ID NO: 29 - RNA, 57 nt, Human immunodeficiency virus
agcagcuaccaaugcugcuugugccuggcuagaagcacaagaggaggaggagguggg

(19) United States
(12) Patent Application Publication
(10) Pub. No.: US 2012/0034189 A1
(43) Pub. Date: Feb. 9, 2012

(54) MEANS AND METHODS FOR DURABLE INHIBITION OF PATHOGENS

(76) Inventors: Karin J. von Eije, Amsterdam (NL); Benjamin Berkhout, Amsterdam (NL); Olivier Ter Brake, Amsterdam (NL)

(21) Appl. No.: 13/203,519

(22) PCT Filed: Feb. 26, 2010
(86) PCT No.: PCT/NL2010/050098

§ 371 Date: Oct. 18, 2011

(30) Foreign Application Priority Data
Feb. 27, 2009 (EP) 09153919.7

(51) Int. Cl. A61K 48/00; C12Q 1/02; A61P 31/12; C12N 5/10; A61K 31/7088; A61P 31/18; C12Q 1/68; C12N 7/06

(52) U.S. Cl. 424/93.2; 435/6.15; 435/29; 435/238; 435/375; 514/44 R

(57) ABSTRACT

The invention provides improved nucleic acids for anti-HIV therapy. The invention further provides selection methods which are capable of predicting already at an early stage of development whether promising anti-pathogenic candidate compounds will be suitable for therapeutic use in vivo.

The invention relates to the fields of biology and medicine.

Infections by pathogenic micro-organisms are an important cause of discomfort, disease and mortality worldwide. During the last decades, much research has been dedicated to prevention and treatment of infections by microbial pathogens such as for instance bacteria, viruses, fungi, worms, parasites and protozoa. Promising therapies have meanwhile been developed, such as for instance antiviral therapies and antibiotic-based therapies. However, such therapies not always remain effective, for instance due to the emergence of escape mutants and/or resistant strains. For instance, although it has become possible to suppress replication and spreading of human immunodeficiency virus (HIV) in an infected individual for a prolonged time, escape mutants nevertheless frequently evolve. Hence, there is an ongoing need to develop new, alternative therapies, particularly anti-HIV therapies.

A currently used approach for the screening for potentially new therapeutic compounds is the use of in vitro assays wherein a pathogen of interest is confronted with many candidate compounds to determine their effect upon the pathogen. Candidate compounds capable of inhibiting functioning, growth, replication and/or proliferation of the pathogen are considered promising lead compounds. Unfortunately, during clinical trials, many promising lead compounds turn out not to be suitable for therapeutic use in humans. For instance, the lead compound appears not to be (sufficiently) effective in vivo and/or the lead compound appears to involve too many negative side effects, rendering it unsuitable for medicinal use. When a promising lead compound ultimately appears to be unsuitable, it often involves a costly loss of materials, test animals, time and money. Efficient selection methods for testing candidate compounds at early phases are therefore important.

It is an object of the present invention to provide novel compositions which are particularly suitable for anti-HIV therapeutic/prophylactic use in animals, preferably humans. It is a further object to provide novel methods for distinguishing candidate compounds which, although effective in vitro, will not be suitable (enough) for therapeutic use, from compounds which have a higher chance of being suitable for therapeutic use in animals and/or humans.

The present invention provides novel combinations of at least three nucleic acid sequences having at least 70% complementarity to specific HIV sequences. According to the present invention, these novel selections of HIV-specific sequences have improved characteristics as compared to currently known HIV therapeutics. For instance, long-lasting protection against HIV is provided while negative side effects are better avoided. Therefore, the novel combinations according to the present invention are more suitable for use as a medicament as compared to other anti-HIV compounds known in the art.

The combinations according to the present invention have been obtained by novel selection procedures, which are outlined in more detail below. The selection methods according to the present invention are capable of predicting already at an early stage of development whether promising lead compounds will be suitable for therapeutic use in vivo, or whether they will cause negative effects such as for instance cell death/apoptosis of the treated cells in the patient. This is determined long before clinical trials are at issue. The nucleic acid combinations and the selection processes according to the present invention have not been described before. Contrary, the present invention teaches against the use of various published anti-HIV constructs, such as for instance the Gag5-containing shRNA constructs described in Ter Brake et al, 2006 (Mol. Ther. Vol 14, No. 6, 883-892) and Ter Brake et al, 2008 (Mol. Ther, Vol 16, No. 3, 557-564). A combination according to the present invention does not contain a nucleic acid sequence complementary to Gag5, because the presence of Gag5 seriously hampers cell function and will therefore involve negative side effects, as described in more detail in the Examples. Gag5 is also called G5.

Accordingly, the present invention provides a compound or a composition or a kit of parts, the compound, composition or kit of parts comprising one or more nucleic acid molecule(s) comprising at least three nucleic acid sequences, each nucleic acid sequence having a length of at least 15 nucleotides, said at least three nucleic acid sequences being selected from the group consisting of a nucleic acid sequence which is at least 75% complementary to a sequence of Table 1 (L2, L3, L4, L5 or L8) and a nucleic acid sequence which is at least 70% complementary to a sequence of Table 2 (P47, P44 or P45) and a nucleic acid sequence which is at least 70% complementary to the sequence ACAGGAGCAGAUGAUACAG (P1) and a nucleic acid sequence which is at least 70% complementary to the sequence AUGGCAGGAAGAAGCGGAG (RT5), wherein said one or more nucleic acid molecule(s) do not comprise a nucleic acid sequence which is complementary to the sequence GAAGAAAUGAUGACAGCAU (G5).

The present invention provides novel therapeutic compositions against HIV which are based on at least three nucleic acid sequences which are at least 70% or at least 75% complementary to specific HIV sequences. Table 1 lists several specific HIV leader sequences and Table 2 lists several specific HIV polymerase sequences. According to the present invention, nucleic acid sequences which are at least 75% complementary to a sequence of Table 1 and/or which are at least 70% complementary to a sequence of Table 2, as well as nucleic acid sequences which are at least 70% complementary to the above mentioned P1 and RT5 sequences, are more suitable for use as a medicament as compared to currently known anti-HIV nucleic acid constructs. The nucleic acid sequences according to the present invention provide long lasting protection against HIV replication while involving fewer side effects, amongst other things as a result of the absence of a nucleic acid sequence which is complementary to the HIV G5 sequence, as outlined in more detail below. The sequences of Tables 1 and 2 are conserved sequences, since they have 100% identity with at least 75% of all HIV sequences currently available in the Los Alamos database. Likewise, the above mentioned HIV P1 sequence and HIV RT5 sequence are conserved. As a result, the combinations according to the invention are useful against a wide variety of HIV strains.

A combination according to the present invention comprises, as an active component against HIV, at least three nucleic acid sequences which are at least 70% or at least 75% complementary to the above mentioned specific HIV sequences. The nucleic acid sequences according to the present invention may be present in one compound, such as for instance in a single vector. Alternatively, said nucleic acid sequences are present in several compounds. For instance, at least three separate nucleic acid constructs are used. Preferably, however, use is made of a single vector harbouring the capability to express all nucleic acid sequences to be used. The use of a single vector for instance ensures that all nucleic acid sequences are present in transfected or transduced host cells. The use of separate nucleic acid constructs involves the risk that several cells contain one or more constructs, but lack another construct, hence resulting in niches in which resistance may develop.

Any combination of at least three nucleic acid sequences with a length of at least 15 nucleotides which are at least 70% or at least 75% complementary to the above mentioned specific HIV sequences is encompassed by the present invention. If a nucleic acid sequence with at least 75% complementarity to a sequence of Table 1 is present, it is preferred that at least two other nucleic acid sequences have complementarity to HIV sequences other than those depicted in Table 1. Likewise, if a nucleic acid sequence with at least 70% complementarity to a sequence of Table 2 is present, it is preferred that at least two other nucleic acid sequences have complementarity to HIV sequences other than those depicted in Table 2. This way, a particularly broad, long lasting protection against HIV is provided. In a preferred embodiment a method according to the invention is therefore provided with the proviso that, if said nucleic acid molecule(s) comprise(s) a nucleic acid sequence which is at least 75% complementary to a sequence of Table 1, then said nucleic acid molecule(s) comprise(s) at least two other nucleic acid sequences which are not at least 75% complementary to a sequence of Table 1 and with the proviso that, if said nucleic acid molecule(s) comprise(s) a nucleic acid sequence which is at least 70% complementary to a sequence of Table 2, then said nucleic acid molecule(s) comprise(s) at least two other nucleic acid sequences which are not at least 70% complementary to a sequence of Table 2.

As used herein, a compound or a composition or a kit of parts comprising one or more nucleic acid molecule(s) comprising at least three nucleic acid sequences according to the invention is also referred to as a combination according to the invention.

In one embodiment, a combination according to the present invention comprises a nucleic acid sequence with a length of at least 15 nucleotides which is at least 75% complementary to a sequence of Table 1, and a nucleic acid sequence with a length of at least 15 nucleotides which is at least 70% complementary to a sequence of Table 2, and a nucleic acid sequence with a length of at least 15 nucleotides which is at least 70% complementary to the above mentioned HIV P1 sequence (ACAGGAGCAGAUGAUACAG). In another embodiment, a combination according to the present invention comprises a nucleic acid sequence with a length of at least 15 nucleotides which is at least 75% complementary to a sequence of Table 1, and a nucleic acid sequence with a length of at least 15 nucleotides which is at least 70% complementary to a sequence of Table 2, and a nucleic acid sequence with a length of at least 15 nucleotides which is at least 70% complementary to the above mentioned HIV RT5 sequence (AUGGCAGGAAGAAGCGGAG). In yet another embodiment, a combination according to the present invention comprises a nucleic acid sequence with a length of at least 15 nucleotides which is at least 75% complementary to a sequence of Table 1, and a nucleic acid sequence with a length of at least 15 nucleotides which is at least 70% complementary to the above mentioned HIV P1 sequence (ACAGGAGCAGAUGAUACAG), and a nucleic acid sequence with a length of at least 15 nucleotides which is at least 70% complementary to the above mentioned HIV RT5 sequence (AUGGCAGGAAGAAGCGGAG). In yet another embodiment, a combination according to the present invention comprises a nucleic acid sequence with a length of at least 15 nucleotides which is at least 70% complementary to a sequence of Table 2, and a nucleic acid sequence with a length of at least 15 nucleotides which is at least 70% complementary to the above mentioned HIV P1 sequence (ACAGGAGCAGAUGAUACAG), and a nucleic acid sequence with a length of at least 15 nucleotides which is at least 70% complementary to the above mentioned HIV RT5 sequence (AUGGCAGGAAGAAGCGGAG). The above mentioned combinations are preferred because they target different HIV sequences which are located sufficiently far from each other. Hence, if one mutation in a wild type HIV genome evolves, it has an impact on the ORF(s) in that region only, while not affecting the other ORF(s) that are also targeted thus reducing the chance of cross resistance. In addition, because many different ORF(s) of the HIV-1 genome are targeted, resistance mutations will affect many different ORF(s) of the virus that may impair de virus significantly.

As used herein, a nucleic acid molecule or nucleic acid sequence of the invention preferably comprises a chain of nucleotides, more preferably DNA and/or RNA. In one preferred embodiment a nucleic acid molecule or nucleic acid sequence of the invention comprises RNA in order to use RNA interference to degrade target RNA, as explained below. In other embodiments a nucleic acid molecule or nucleic acid sequence of the invention comprises other kinds of nucleic acid structures such as for instance a DNA/RNA helix, peptide nucleic acid (PNA), locked nucleic acid (LNA) and/or a ribozyme. Hence, the term “nucleic acid molecule” or “nucleic acid sequence” also encompasses a chain comprising non-natural nucleotides, modified nucleotides and/or non-nucleotide building blocks which exhibit the same function as natural nucleotides. As used herein, such non-natural nucleotides, modified nucleotides and/or non-nucleotide building blocks are referred to with the term “nucleotide analogues”.

Preferably, a combination of at least three nucleic acid sequences according to the present invention comprises a nucleic acid sequence which is at least 75% complementary to the L4 sequence of Table 1 and/or a nucleic acid sequence which is at least 70% complementary to the P47 sequence of Table 2. As explained in more detail in the Examples, such nucleic acid sequences are particularly well capable of inhibiting HIV for a prolonged time, while negative side effects are particularly well avoided. A preferred embodiment of the invention therefore provides a compound or a composition or a kit of parts comprising one or more nucleic acid molecule(s) comprising at least three nucleic acid sequences, each nucleic acid sequence having a length of at least 15 nucleotides, said at least three nucleic acid sequences being selected from the group consisting of a nucleic acid sequence which is at least 75% complementary to the sequence GAGAGAGAUGGGUGCGAGA (L4) and a nucleic acid sequence which is at least 70% complementary to the sequence ACAGGAGCAGAUGAUACAG (P1) and a nucleic acid sequence which is at least 70% complementary to the sequence GUGAAGGGGCAGUAGUAAU (P47) and a nucleic acid sequence which is at least 70% complementary to the sequence AUGGCAGGAAGAAGCGGAG (RT5).

A nucleic acid sequence is at least 70% or at least 75% complementary to any of the above mentioned HIV sequences if said nucleic acid sequence has at least 70% or at least 75% sequence identity to the complement of said HIV sequence. A complement of said HIV sequence is a sequence (usually called an anti-sense sequence) which is 100% complementary to said HIV sequence. Hence, a combination according to the present invention comprises at least three nucleic acid sequences which have at least 75% sequence identity to the complements of the HIV sequences depicted in Table 1 and/or which have at least 70% sequence identity to the complements of the HIV sequences selected from the group consisting of Table 2, the above mentioned HIV P1 sequence and the above mentioned RT5 sequence. In order to improve HIV specificity and/or anti-HIV activity, especially when RNA interference is used, said at least three nucleic acid sequences preferably are at least 80%, more preferably at least 85%, more preferably at least 90% complementary to any of the above mentioned HIV sequences. This means that said at least three nucleic acid sequences preferably have at least 80%, more preferably at least 85%, more preferably at least 90% sequence identity to the complements of HIV sequences selected from the group consisting of Table 1, Table 2, the above mentioned HIV P1 sequence and the above mentioned RT5 sequence.

The term “% sequence identity” is defined herein as the percentage of nucleotides or nucleotide analogues in a nucleic acid sequence according to the invention that is identical with the corresponding nucleotides in a reference sequence after aligning the two sequences and introducing gaps, if necessary, to achieve the maximum percent identity. Hence, in case a reference sequence is longer than a nucleic acid according to the invention, additional nucleotides in the reference sequence, against which a nucleic acid sequence according to the invention is not directed, are not taken into account for determining sequence identity. Methods and computer programs for alignment are well known in the art. One computer program which may be used or adapted for determining the percentage of sequence identity is “Align 2”, authored by Genentech, Inc., which was filed with user documentation in the United States Copyright Office, Washington, D.C. 20559, on Dec. 10, 1991.

A combination according to the present invention comprises at least three nucleic acid sequences having at least 70% or at least 75% complementarity to specific HIV sequences selected from the group consisting of Table 1, Table 2, the above mentioned HIV P1 sequence and the above mentioned RT5 sequence. In one preferred embodiment a combination according to the invention is provided which contains three nucleic acid sequences according to the present invention as sole active anti-HIV component. Such combination is on the one hand very effective in providing durable inhibition of HIV, and on the other hand capable of being efficiently produced, transfected and/or transduced. Moreover, the lesser nucleic acid sequences, the lower the risk of side effects is. One preferred embodiment therefore provides a compound or a composition or a kit of parts according to the invention containing, as the sole active component against HIV, three nucleic acid sequences with a length of at least 15 nucleotides, selected from the group consisting of a nucleic acid sequence which is at least 75% complementary to a sequence of Table 1 (L2, L3, L4, L5 or L8) and a nucleic acid sequence which is at least 70% complementary to a sequence of Table 2 (P47, P44 or P45) and a nucleic acid sequence which is at least 70% complementary to the sequence ACAGGAGCAGAUGAUACAG (P1) and a nucleic acid sequence which is at least 70% complementary to the sequence AUGGCAGGAAGAAGCGGAG (RT5). Such combinations often contain additional nucleic acid sequences which are not specifically directed against HIV, such as promoters and/or other regulatory sequences and/or vector-derived sequences. Preferably, said three nucleic acid sequences are at least 80%, more preferably at least 85%, more preferably at least 90% complementary to said HIV sequences. Furthermore, a combination of nucleic acid sequences according to the invention is optionally combined with other anti-HIV therapeutics, such as for instance reverse transcriptase inhibitors (NRTI's and NNRTI's, an example of such an NRTI is zidovudine (AZT)), and protease inhibitors, (from a combination of these three classes of drugs consist HAART). Other suitable drugs are entry inhibitors and integrase inhibitors. In one embodiment, a combination of nucleic acid sequences according to the invention is combined with at least one other drug selected from the group consisting of Zidovudine (AZT), Abacavir, Didanosine (=ddI), Lamivudine (=3TC), Stavudine (=d4T), Tenofovirdisoproxil, Enfuvirtide, Emtricitabine, Amprenavir, atazanavir, darunavirfosamprenavir, indinavir, lopinavir, nelfinavir, ritonavir, saquinavir, tipranavir, Amprenavir, Fosamprenavir, Atazanavir, Tipranavir, Nevirapine and efavirenz.

As explained before, a particularly preferred sequence of Table 1 to be targeted is L4. Furthermore, a particularly preferred sequence of Table 2 to be targeted is P47. In such case, use is preferably made of a nucleic acid sequence which is at least 75% complementary to the L4 sequence of Table 1 and/or a nucleic acid sequence which is at least 70% complementary to the P47 sequence of Table 2. In one embodiment, therefore, a compound or a composition or a kit of parts according to the invention contains, as the sole component against HIV, three nucleic acid sequences with a length of at least 15 nucleotides, selected from the group consisting of a nucleic acid sequence which is at least 75% complementary to the sequence GAGAGAGAUGGGUGCGAGA (L4) and a nucleic acid sequence which is at least 70% complementary to the sequence ACAGGAGCAGAUGAUACAG (P1) and a nucleic acid sequence which is at least 70% complementary to the sequence GUGAAGGGGCAGUAGUAAU (P47) and a nucleic acid sequence which is at least 70% complementary to the sequence AUGGCAGGAAGAAGCGGAG (RT5). As said before, such combinations often contain additional nucleic acid sequences, such as promoters, which are not specifically directed against HIV. Preferably, said three nucleic acid sequences are at least 80%, more preferably at least 85%, more preferably at least 90% complementary to said HIV sequences. In one embodiment, sequences are used which are 100% complementary to said HIV sequences.

In one particularly preferred embodiment a combination is made of at least three nucleic acid sequences which are, in combination, at least 75% complementary to L4 and at least 70% complementary to P1 and P47. Such combination is particularly suitable for durable inhibition of HIV, while avoiding negative side effects. This embodiment thus provides a compound or a composition or a kit of parts according to the invention comprising a nucleic acid sequence with a length of at least 15 nucleotides which is at least 75% complementary to the sequence GAGAGAGAUGGGUGCGAGA (L4) and a nucleic acid sequence with a length of at least 15 nucleotides which is at least 70% complementary to the sequence ACAGGAGCAGAUGAUACAG (P1) and a nucleic acid sequence with a length of at least 15 nucleotides which is at least 70% complementary to the sequence GUGAAGGGGCAGUAGUAAU (P47).

In another embodiment, a combination is made of at least three nucleic acid sequences which are, in combination, at least 75% complementary to L4 and at least 70% complementary to P1 and RT5. Such combination is also particularly suitable for durable inhibition of HIV, while avoiding negative side effects. Further provided is therefore a compound or a composition or a kit of parts according to the invention comprising a nucleic acid sequence with a length of at least 15 nucleotides which is at least 75% complementary to the sequence GAGAGAGAUGGGUGCGAGA (L4) and a nucleic acid sequence with a length of at least 15 nucleotides which is at least 70% complementary to the sequence ACAGGAGCAGAUGAUACAG (P1) and a nucleic acid sequence with a length of at least 15 nucleotides which is at least 70% complementary to the sequence AUGGCAGGAAGAAGCGGAG (RT5).

In yet another embodiment, a combination is made of at least three nucleic acid sequences which are, in combination, at least 75% complementary to L4 and at least 70% complementary to P47 and RT5. Such combination is also particularly suitable for durable inhibition of HIV, while avoiding negative side effects. Further provided is therefore a compound or a composition or a kit of parts according to the invention comprising a nucleic acid sequence with a length of at least 15 nucleotides which is at least 75% complementary to the sequence GAGAGAGAUGGGUGCGAGA (L4) and a nucleic acid sequence with a length of at least 15 nucleotides which is at least 70% complementary to the sequence GUGAAGGGGCAGUAGUAAU (P47) and a nucleic acid sequence with a length of at least 15 nucleotides which is at least 70% complementary to the sequence AUGGCAGGAAGAAGCGGAG (RT5).

A combination which contains three nucleic acid sequences as the sole component against HIV, which nucleic acid sequences are at least 70% complementary to P1, P47 and RT5, is also provided. Such combination according to the invention is more suitable for use as a medicament than currently known constructs, such as for instance the construct described in Ter Brake et al, 2008, because negative side effects are better avoided due to the absence of a Gag5 sequence, contrary to Ter Brake et al 2008. According to the present invention, the Gag5 sequence diminishes cell function and therefore involves negative side effects. The use of a Gag5 sequence is therefore preferably avoided. Further provided is therefore a compound or a composition or a kit of parts according to the invention containing, as the sole component against HIV, a nucleic acid sequence with a length of at least 15 nucleotides which is at least 70% complementary to the sequence ACAGGAGCAGAUGAUACAG (P1) and a nucleic acid sequence with a length of at least 15 nucleotides which is at least 70% complementary to the sequence GUGAAGGGGCAGUAGUAAU (P47) and a nucleic acid sequence with a length of at least 15 nucleotides which is at least 70% complementary to the sequence AUGGCAGGAAGAAGCGGAG (RT5).

Preferably, the above mentioned nucleic acid sequences are at least 80%, more preferably at least 85%, more preferably at least 90% complementary to said HIV sequences. In one embodiment, sequences are used which are 100% complementary to the above mentioned HIV sequences. One further preferred embodiment therefore provides a compound, composition or kit of parts according to the invention, wherein said nucleic acid molecule(s) contains as the sole component against HIV three nucleic acid sequences, each of said nucleic acid sequences being selected from the group consisting of a nucleic acid sequence which is complementary to the sequence GAGAGAGAUGGGUGCGAGA (L4) and a nucleic acid sequence which is complementary to the sequence ACAGGAGCAGAUGAUACAG (P1) and a nucleic acid sequence which is complementary to the sequence GUGAAGGGGCAGUAGUAAU (P47) and a nucleic acid sequence which is complementary to the sequence AUGGCAGGAAGAAGCGGAG (RT5). Said three nucleic acid sequences each have a length of at least 15 nucleotides.

As said before, the above mentioned combinations often contain additional nucleic acid sequences which are not specifically directed against HIV, such as promoters and/or other regulatory sequences and/or vector-derived sequences.

In another embodiment a compound or a composition or a kit of parts according to the invention is provided which comprises at least four of the above mentioned nucleic acid sequences. Such combination is especially dedicated to long term protection against HIV replication, while negative side effects are diminished or avoided as compared to other, currently known, anti-HIV nucleic acid constructs. Further provided is therefore a compound or a composition or a kit of parts according to the invention comprising a nucleic acid sequence with a length of at least 15 nucleotides which is at least 75% complementary to a sequence of Table 1 (L2, L3, L4, L5 or L8) and a nucleic acid sequence with a length of at least 15 nucleotides which is at least 70% complementary to a sequence of Table 2 (P47, P44 or P45) and a nucleic acid sequence with a length of at least 15 nucleotides which is at least 70% complementary to the sequence ACAGGAGCAGAUGAUACAG (P1) and a nucleic acid sequence with a length of at least 15 nucleotides which is at least 70% complementary to the sequence AUGGCAGGAAGAAGCGGAG (RT5).

As explained before, a particularly preferred sequence of Table 1 to be targeted is L4. Furthermore, a particularly preferred sequence of Table 2 to be targeted is P47. In such case, use is preferably made of a nucleic acid sequence which is at least 75% complementary to the L4 sequence of Table 1 and a nucleic acid sequence which is at least 70% complementary to the P47 sequence of Table 2. Further provided is therefore a compound or a composition or a kit of parts according to the invention, comprising a nucleic acid sequence with a length of at least 15 nucleotides which is at least 75% complementary to the sequence GAGAGAGAUGGGUGCGAGA (L4) and a nucleic acid sequence with a length of at least 15 nucleotides which is at least 70% complementary to the sequence ACAGGAGCAGAUGAUACAG (P1) and a nucleic acid sequence with a length of at least 15 nucleotides which is at least 70% complementary to the sequence GUGAAGGGGCAGUAGUAAU (P47) and a nucleic acid sequence with a length of at least 15 nucleotides which is at least 70% complementary to the sequence AUGGCAGGAAGAAGCGGAG (RT5). Preferably, said four nucleic acid sequences are at least 80%, more preferably at least 85%, more preferably at least 90% complementary to said HIV sequences. In one embodiment, nucleic acid sequences are used which are 100% complementary to said HIV sequences.

Combinations according to the present invention may comprise more than four nucleic acid sequences, in order to improve long-lasting protection. In one embodiment, however, said four above mentioned nucleic acid sequences are the sole active ingredient against HIV, although they may contain additional nucleic acid sequences which are not specifically directed against HIV, such as promoters and/or other regulatory sequences and/or vector-derived sequences.

In one preferred embodiment, a nucleic acid sequence according to the present invention has a length of at least 16 nucleotides. More preferably, a nucleic acid sequence according to the present invention has a length of at least 17 nucleotides. In a further preferred embodiment, a nucleic acid sequence according to the present invention has a length of at least 18 nucleotides.

In another embodiment, only sequences are used which are 100% complementary to the above mentioned HIV sequences. Further provided is therefore a compound or a composition or a kit of parts according to the invention, comprising a nucleic acid sequence which is complementary to a sequence of Table 1 (L2, L3, L4, L5 or L8) and/or a nucleic acid sequence which is complementary to a sequence of Table 2 (P47, P44 or P45) and/or a nucleic acid sequence which is complementary to the sequence ACAGGAGCAGAUGAUACAG (P1) and/or a nucleic acid sequence which is complementary to the sequence AUGGCAGGAAGAAGCGGAG (RT5).

Preferably, said combination comprises a nucleic acid sequence which is complementary to the sequence GAGAGAGAUGGGUGCGAGA (L4) and/or a nucleic acid sequence which is complementary to the sequence GUGAAGGGGCAGUAGUAAU (P47) and/or a nucleic acid sequence which is complementary to the sequence ACAGGAGCAGAUGAUACAG (P1) and/or a nucleic acid sequence which is complementary to the sequence AUGGCAGGAAGAAGCGGAG (RT5). In one embodiment, four nucleic acid sequences are used as the active component against HIV. Further provided is therefore a compound or a composition or a kit of parts according to the invention comprising a nucleic acid sequence which is complementary to the sequence GAGAGAGAUGGGUGCGAGA (L4) and a nucleic acid sequence which is complementary to the sequence GUGAAGGGGCAGUAGUAAU (P47) and a nucleic acid sequence which is complementary to the sequence ACAGGAGCAGAUGAUACAG (P1) and a nucleic acid sequence which is complementary to the sequence AUGGCAGGAAGAAGCGGAG (RT5).

The present invention provides novel, preferred combinations of at least three nucleic acid sequences having at least 70% or at least 75% complementarity to specific HIV sequences. Various kinds of nucleic acid sequences capable of targeting said HIV sequences are suitable for a combination according to the invention. For instance, antisense DNA oligonucleotides are used. Moreover, ribozymes capable of catalyzing cleavage of target RNAs are suitable. A preferred method for gene silencing is, however, RNA interference (RNAi). RNA interference is a sequence-specific RNA degradation process in the cytoplasm of eukaryotic cells that is induced by double stranded RNA. This RNA-silencing mechanism, which was first described in Caenorhabditis elegans and Drosophila melanogaster has many similarities with post-transcriptional gene silencing in plants. RNAi and related RNA silencing mechanisms are believed to act as a natural defense against incoming viruses and the expression of transposable elements. Besides such antiviral function of RNAi there is evidence that RNAi plays an important role in regulating cellular gene expression. These features have characterized RNAi both as an ancient and fundamentally important mechanism in eukaryotic cell biology.

RNAi is induced in mammalian cells by double stranded RNAs which are recognised by a DICER enzyme. Processing of double stranded RNA by DICER yields so-called small interfering RNAs (siRNAs) of approximately 19-23 base pairs (bp) with two nucleotides overhangs. These siRNAs associate with various proteins to form the RNA-induced silencing complex (RISC), harbouring nuclease and helicase activity. The antisense strand of the siRNA guides the RISC to the complementary target RNA, and the nuclease component cleaves the target RNA in a sequence-specific manner. Hence, double stranded RNA is capable of inducing degradation of the homologous single stranded RNA in a host cell.

The antiviral capacity of RNA silencing has been used as a tool to generate virus resistance in plants. RNA-mediated virus resistance was obtained by expression of untranslatable viral coat-protein RNAs in transgenic plants. Expression of hairpin RNAs corresponding to viral sequences induced virus resistance in almost 100% of the transgenic plants. In analogy with RNA-mediated virus resistance in plants, RNAi technology is currently being used to inhibit viral replication in animal cells. Several results have been obtained with RNAi against several animal viruses both in in vitro and in vivo settings. For instance, Das et al have demonstrated that inhibition of HIV-1 replication through RNAi is possible in stably transduced cells. However, HIV-1 escape mutants that were no longer inhibited appeared after several weeks of culture (Das et al. Journal of Virology, Vol 78, No. 5 (2004) pages 2601-2605). Contrary, combinations according to the present invention provide long-lasting protection against HIV and are therefore preferred.

In one embodiment of the present invention, a host cell is provided with double stranded RNA by transfection of double stranded RNA molecules such as siRNA. siRNAs are for instance chemically or enzymatically synthesized. If long-term suppression of an organism is required, it is preferred to provide a cell with an expression cassette so that a double stranded RNA is continuously produced by the host cell. Intracellular synthesis of a siRNA is for instance achieved by expression of separate sense and antisense RNA fragments, which together form a dsRNA. Further provided herein is, therefore, a compound or a composition or a kit of parts according to the present invention comprising at least one small interfering RNA (siRNA). More preferably, a compound or composition or kit of parts according to the invention comprises at least two siRNAs or, even more preferably, at least three siRNAs.

More preferably however, a short hairpin RNA (shRNA) is expressed by the host cell. Activation of the RNAi machinery by intracellular expression, preferably expression of an shRNA, has the advantage over transfection of synthetic siRNA that there is a constitutive transcription ensuring a basal level of RNAi activity. A short hairpin RNA comprises at least one base paired stem (also called a base paired duplex) comprising the sense and antisense strand of a siRNA, preferably with a length of between 15-40 nucleotides, more preferably with a length of between 15-30 nucleotides, more preferably with a length of between 19-25 nucleotides, most preferably with a length of between 19-23 nucleotides, preferably with 2 nucleotides overhang. The sense and antisense strand of a shRNA are linked with a small loop.

In one embodiment single short hairpins are used. If single short hairpins are used, the sense and antisense strands are joined through a loop of several nucleotides. A single shRNA of the invention preferably comprises a loop with a length of about 2 to 15 nucleotides. More preferably said loop has a length of about 3 to 10 nucleotides, most preferably about 5 to 9 nucleotides. In one embodiment said loop comprises hairpin-stabilizing tetraloop sequences.

In one preferred embodiment, multiple shRNA expression cassettes are inserted into a single vector. The units are clustered (parallel or antiparallel orientation) or inserted at different positions of a vector. In a preferred embodiment multiple short hairpin RNAs are combined in a single expression construct. For instance, a stacked multiple shRNA structure is used wherein the dsRNA sequences are carefully arranged to allow efficient processing to generate the individual siRNA units, or a branched, tRNA-like multiple shRNA structure is used wherein each branch is a separate shRNA. The efficiency of multiple shRNA constructs is preferably optimized by inclusion of signals capable of determining the intracellular location and stability of the RNA transcript. A multi-shRNA transcript is preferably made from at least one polymerase II or polymerase III promoter. Examples of multi-shRNA transcripts are shown in FIG. 8 and in Anderson et al, Oligonucleotides 13:303-312 (2003). FIG. 9 provides a schematic overview of RNAi interference with shRNA.

Further provided herein is, therefore, a compound or a composition or a kit of parts according to the present invention comprising at least one short hairpin RNA (shRNA). More preferably, a compound or composition or kit of parts according to the invention comprises at least two shRNAs. Even more preferably, a compound or composition or kit of parts according to the invention comprises at least three shRNAs. As described above, a combination according to the present invention preferably comprises a multiple short hairpin construct. The basepaired duplex of an extended shRNA of the invention is preferably destabilized. This is for instance performed by inclusion of weak G-U base pairs and/or mispairs, and/or by destabilizing bulge or internal loop elements by modification of the sense strand.

Whereas the standard expression strategy for a shRNA transcript is the use of a Polymerase III (Pol III) promoter (e.g. H1 or U6), the expression of a longer transcript encoding multiple shRNAs is preferably optimized. This for instance includes the use of a Polymerase II (Pol II) promoter or the T7 expression system for cytoplasmic RNA expression. In one preferred embodiment, different promoters are used for each shRNA in a multiple short hairpin construct, such as for instance described in Ter Brake et al, 2008. In a preferred embodiment, therefore, at least two but preferably at least three different promoters are used. A promoter used in a short hairpin construct of the present invention is for instance constitutively expressed, either in all cell types/tissues or in a cell/tissue-specific manner. In one embodiment, at least one inducible promoter is used so that the amount of expression of double stranded RNA is regulated.

In another preferred embodiment, a combination of at least three nucleic acid sequences according to the present invention comprises at least one microRNA (miRNA) structure.

MicroRNAs (miRNAs) are small RNA molecules encoded in the genomes of plants and animals and viruses. They can be present as individual miRNAs or be present in transcripts encoding multiple miRNAs. They can be present within introns of protein-coding genes. These highly conserved RNAs with a length of approximately 21-23 nucleotides usually regulate the expression of genes by binding to the 3′-untranslated regions (3′-UTRs) of specific mRNAs. Each miRNA is thought to regulate multiple genes, and since hundreds of miRNA sequences are predicted to be present in higher eukaryotes the potential regulatory circuitry afforded by miRNA is enormous. Several research groups have provided evidence that miRNAs act as key regulators of processes as diverse as early development, cell proliferation and cell death, apoptosis, fat metabolism, and cell differentiation. There is speculation that in higher eukaryotes, the role of miRNAs in regulating gene expression could be as important as that of transcription factors.

miRNAs are transcribed by RNA polymerase II and III. The primary transcripts, which generally have a length of several kilobases, are called pri-miRNAs. Pri-miRNAs are processed in the cell nucleus to shorter, 70-100 nucleotide stem-loop structures known as pre-miRNAs. This processing is performed in animals by the RNase III endonuclease Drosha. Pre-miRNAs are subsequently transported into the cytoplasm, where they are processed to miRNAs with a length of 21-23 nucleotides by the RNase III endonuclease DICER. One strand of the miRNA duplex is subsequently incorporated into the RNA-induced silencing complex (RISC). As part of the RISC, gene expression of a target gene is counteracted by inhibiting translation and/or by cleaving mRNA. FIG. 10 provides a non-limiting, schematic overview.

Hence, like siRNAs and shRNAs, miRNA structures are capable of inducing cleavage of target RNA using the RISC complex. In one preferred embodiment, therefore, a compound or a composition or a kit of parts according to the present invention comprises at least one miRNA structure. More preferably, a compound or composition or kit of parts according to the invention comprises at least two miRNA structures. Most preferably, a compound or composition or kit of parts according to the invention comprises at least three miRNA structures.

As used herein, the term “miRNA structure” encompasses non natural miRNA structures, which have been designed and/or generated by man. Such non natural miRNA structures for instance have a length of between 15-23 nucleotides. Alternatively, such non natural miRNA structure comprises a longer nucleotide sequence, which is capable of being processed by Drosha and/or DICER resulting in RNA molecules of about 21-23 nucleotides. Such non natural miRNA structure preferably consists of RNA. Alternatively, however, another kind of nucleic acid is used, such as DNA, which is converted into RNA at a later stage. A non natural miRNA structure according to the invention is, like natural miRNA, capable of counteracting expression of a target gene (optionally after being converted into RNA and/or after being processed by Drosha and/or DICER). In one embodiment a miRNA structure is designed based on a natural miRNA sequence. For instance, an existing miRNA sequence is at least partly altered. Alternatively, a nucleic acid molecule is designed de novo. In both cases care is preferably taken in order to ensure that the resulting structure is suitable for processing in vivo by the miRNA machinery.

siRNAs, shRNAs and miRNA structures are thus the preferred kinds of nucleic acid sequences in combinations according to the invention. A preferred embodiment therefore provides a compound or a composition or a kit of parts according to the present invention wherein said at least three nucleic acid sequences comprise at least one shRNA and/or at least one siRNA and/or at least one miRNA structure, preferably at least two shRNAs and/or at least two siRNAs and/or at least two miRNA structures, most preferably at least three shRNAs and/or at least three siRNAs and/or at least three miRNA structures.

The nucleic acid combinations according to the present invention provide long-lasting protection against HIV while negative side effects are better avoided, as compared to currently known anti-HIV nucleic acid constructs. The combinations according to the present invention are therefore particularly suitable for therapeutic and/or prophylactic use in animals, particularly in humans. A compound or a composition or a kit of parts according to the invention for use as a medicament is therefore herewith provided. As used herein, the term “medicament” encompasses therapeutic drugs and prophylactic agents. As the combinations according to the invention are suitable for durable inhibition of replication, proliferation and/or spreading of HIV, the invention further provides a compound or a composition or a kit of parts according to the invention for use in a method for durable inhibition of HIV. In one embodiment a combination according to the invention is used for the preparation of an anti-HIV medicament. A use of a compound or composition or kit of parts according to the invention for the preparation of a medicament against HIV is therefore also provided, as well as a compound or a composition or a kit of parts according to the invention for use in the treatment and/or prophylaxis of AIDS, as well as a pharmaceutical composition comprising a combination of nucleic acid sequences according to the invention and a pharmaceutically acceptable diluent, carrier or excipient. Examples of suitable carriers for instance comprise keyhole limpet haemocyanin (KLH), serum albumin (e.g. BSA or RSA) and ovalbumin. In another embodiment, said suitable carrier comprises a solution like for example saline.

Further provided is a gene delivery vehicle or an isolated or recombinant host cell comprising a combination of nucleic acid sequences according to the invention. A gene delivery vehicle is a construct capable of providing a cell with a nucleic acid of interest. Gene delivery vehicles are well known in the art. In one preferred embodiment, said gene delivery vehicle comprises a vector. Suitable vectors for instance comprise adenoviral vectors, adeno-associated viral (AAV) vectors, gamma retroviral vectors and lentiviral vectors. Said vector is preferably a retroviral vector, more preferably a lentiviral vector, more preferably a lentiviral vector based on HIV, because retroviral vectors are particularly well adapted for inserting nucleic acids into a host cell. However, all kinds of gene delivery vehicles are suitable, as long as they are capable of introducing at least one nucleic acid sequence according to the invention into a host cell. In one embodiment, a gene delivery vehicle according to the present invention is a plasmid delivery system (preferably a transposable element), a virus like particle (VLP), a stable nucleic acid lipid particle (SNALP), a cholesterol conjugate, a cationic delivery system, a cationic liposomal delivery system, a cationic polymer, a peptide delivery system, a lipoplex, or a liposome. In one embodiment an aptamer is used.

Transposable elements have emerged as a promising candidate for human non-viral gene-therapy. A non-limiting example is the Sleeping Beauty Transposon™ System (SBTS) that is a non-viral carrier of genetic information that is capable of inserting a nucleic acid sequence into vertebrate (including human) chromosomes in order to confer a new function or replace a defective gene.

Virus like particles contain viral protein(s) derived from the structural proteins of a virus. In some cases these proteins are embedded within a lipid bilayer. These particles resemble the virus from which they were derived but lack viral nucleic acid, meaning that they are not infectious. VLPs used as vaccines are often very effective at eliciting both T cell and B cell immune responses. Also these VLPs are suitable for packaging plasmids or si/sh/miRNAs. By modifications of the viral proteins and linkage of other molecules to the surface of the particle their tropism can be modulated.

Stable nucleic acid-lipid particles are specialized lipid nanoparticles that fully encapsulate and systemically deliver a variety of nucleic acid molecules such as for instance siRNA, aptamers and plasmid DNA. The packaging compounds of the particle can contain cationic lipids, fusogenic lipids and PEG lipids. This particle delivers nucleic acids inside a cell where it has its intended effect.

A cholesterol conjugate is in one preferred embodiment used as follows: short interfering RNAs (siRNAs) are synthesized, chemically modified and labelled on the ‘sense’ strand with cholesterol. The siRNAs are then injected intravenously into an animal, where the cholesterol group enables the siRNAs to be taken up into tissues.

Cellular delivery of siRNA is in one embodiment addressed by the use of cationic macromolecules, the two major classes being lipids and polymers. Examples of cationic vectors are liposomes (lipoplexes) and polyethelyenimine (PEI) (polyplexes).

Peptide delivery systems are based on a class of small cationic peptides capable of passing a cell's membrane and gaining access to the intracellular environment. Importantly, cellular entrance is also permitted for covalently coupled cargo. The cationic nature of these peptides is crucial for their ability to bind and traverse the anionic cellular membrane. Cell penetrating peptides (CPPs) can be used for the delivery of a wide range of macromolecules including RNA interference (RNAi).

A lipoplex comprises a complex of nucleic acids mixed with lipids that spontaneously forms a vesicle capable of containing therapeutic nucleic acids of interest.

A liposome is a vesicle, made out of the same material as a cell membrane. Liposomes can be filled with drugs and they are used to deliver drugs at a site of interest.

Aptamers are oligonucleic acid or peptide molecules that are capable of binding a specific target molecule. Aptamers are usually created by selecting them from a large random sequence pool, but natural aptamers also exist in riboswitches. Aptamers are used for both basic research and clinical purposes as macromolecular drugs. Aptamers can be combined with ribozymes to self-cleave in the presence of their target molecule. These compound molecules have additional research, industrial and clinical applications. More specifically, aptamers can be classified as DNA or RNA aptamers. They normally contain (usually short) strands of oligonucleotides. The other type is a peptide aptamer. Peptide aptamers normally contain a short variable peptide domain, which are often attached at both ends to a protein scaffold. Aptamers can be part of a synthetic siRNA. Alternatively, or additionally, aptamers can be coupled to a carrier.

In yet another embodiment, HIV Tat-mediated transduction is performed.

Further provided is a method for at least in part inhibiting replication and/or proliferation and/or spreading of HIV, comprising contacting said HIV with a compound, or a composition, or a kit of parts, or a pharmaceutical composition, or a gene delivery vehicle according to the invention. Preferably, HIV is counteracted in an animal, more preferably in a human individual. Further provided is therefore a method for at least in part treating or preventing AIDS, comprising administering to a subject suffering from, or at risk of suffering from, AIDS a therapeutically active amount or a prophylactically active amount of a compound, or a composition, or a kit of parts, or a pharmaceutical composition, or a gene delivery vehicle according to the invention. In one preferred embodiment, said subject is diagnosed for HIV infection before administration of a compound or composition or kit of parts or pharmaceutical composition or gene delivery vehicle according to the invention. Such diagnosis is for instance performed by determining whether a sample of said subject comprises HIV-specific nucleic acid or HIV-specific antibodies or HIV-specific T-cells. Said sample preferably comprises a blood sample. If the subject appears to be infected by HIV, a therapeutically active amount of a compound or composition or kit of parts according to the invention, or a therapeutically active amount of a pharmaceutical composition according to the invention, or a therapeutically active amount of a gene delivery vehicle according to the invention, is preferably administered to him or her.

In one embodiment cells from the hematopoietic lineage of an individual, preferably T-cells and/or hematopoietic stem cells and/or hematopoietic progenitor cells, are provided ex vivo with a nucleic acid combination according to the present invention, where after the cells are returned back to the individual. T-cells originating from these cells will be capable of durably resisting HIV replication. A non-limiting example of such therapy is shown in FIG. 11. Further provided is therefore a method for at least in part inhibiting replication and/or proliferation and/or spreading of HIV in an individual, comprising providing cells from the hematopoietic lineage of said individual, preferably T-cells and/or hematopoietic stem cells and/or hematopoietic progenitor cells, with one or more nucleic acid molecule(s) according to the present invention. This results in the presence of T-cells in said individual which are capable of durably resisting HIV replication. Preferably, said cells from the hematopoietic lineage of said individual, preferably T-cells and/or hematopoietic stem cells and/or hematopoietic progenitor cells, are provided with a nucleic acid combination according to the present invention ex vivo. Subsequently, said cells are preferably returned back to the individual. An isolated or recombinant T-cell and/or hematopoietic stem cell and/or hematopoietic progenitor cell comprising one or more nucleic acid molecule(s) according to the present invention is therefore also provided, as well as a T-cell and/or hematopoietic stem cell and/or hematopoietic progenitor cell according to the invention for use as a medicament or prophylactic agent. Such T-cell and/or hematopoietic stem cell and/or hematopoietic progenitor cell is, of course, preferably used as a medicament or prophylactic agent against AIDS.

In another aspect, the present invention provides methods for determining whether a candidate compound, capable of inhibiting a pathogen in vitro, is suitable for development of a medicament for use in vivo. The present invention provides three selection criteria which, separately or combined, are capable of distinguishing between candidate compounds with a low chance of therapeutic suitability and candidate compounds with a higher chance of therapeutic suitability. These selection methods concern a candidate compound's capability of providing long-lasting protection and the chance of negative side effects. The present invention provides selection methods with which compounds which will be unsuitable for medicinal use in animals and/or humans can be rejected at an early stage of research before clinical trials are performed. Such unsuitable compounds are thus no longer tested, which saves costly materials, test animals, time and money and renders the further development stages more effective since only promising compounds are further tested and/or developed. The above described combinations of nucleic acids according to the present invention, which are particularly suitable for anti-HIV therapies in animals and humans, were selected using selection methods according to the present invention.

Accordingly, the invention provides a method for determining whether a candidate compound which is capable of inhibiting a pathogenic micro-organism is suitable for use as a medicament, the method comprising:

introducing said candidate compound into a cell wherein said pathogenic micro-organism, if present, is capable of replicating and/or proliferating and determining whether said cell comprising said candidate compound exhibits between 85% and 115%, preferably between 90% and 110%, most preferably between 95% and 105% cell function as compared to the same kind of cell which has not been provided with said candidate compound, which indicates that said candidate compound is suitable for further testing and/or development of a medicament, and

introducing said candidate compound into a cell wherein said pathogenic micro-organism, if present, is capable of replicating and/or proliferating, and infecting said cell with said pathogenic micro-organism, at a dose which would result in replication of said pathogenic micro-organism in the same kind of cells which do not comprise said candidate compound, and determining whether replication and/or proliferation of said pathogenic micro-organism in cells comprising said candidate compound is inhibited during culturing for at least 80 days, which indicates that said candidate compound is suitable for further testing and/or development of a medicament; and/or

introducing said candidate compound into a cell wherein said pathogenic micro-organism, if present, is capable of replicating and/or proliferating, and infecting said cell with said pathogenic micro-organism at a dose which firstly results in inhibition of said pathogenic micro-organism during at least 20 days and subsequently results in replication of said pathogenic micro-organism in said cell, and determining whether at least 80% of the replicating micro-organisms are escape mutants of said pathogenic micro-organisms, which indicates that said candidate compound is suitable for further testing and/or development of a medicament.

A method according to the present invention is preferably performed in vitro. Preferably, a candidate compound is tested which comprises at least one nucleic acid sequence with a length of at least 15 nucleotides which is at least 70% complementary to a genomic sequence of a pathogenic micro-organism. Such nucleic acid sequences, even though effective in vitro, often turn out to be unsuitable for therapies in humans. Preferably, at least one nucleic acid sequence with a length of at least 15 nucleotides which is at least 70% complementary to a genomic sequence of HIV is tested.

In one aspect, it is determined whether a cell comprising a candidate compound exhibits between 85% and 115%, preferably between 90% and 110%, most preferably between 95% and 105% cell function as compared to the same kind of cell which has not been provided with said candidate compound. Surprisingly, it has been established by the present inventors that a nucleic acid sequence which is specific for a pathogenic micro-organism is still capable of seriously hampering normal cell function when administered in low doses which are indicative for therapeutic doses in human therapies. This holds even true when RNA interference is used, even though their mode of action is so specific that even a single mismatch between the RNA and its target sequence inhibits their capability of cleaving the target sequence. Hence, even though a single mismatch inhibits their cleaving activity, and even when relatively low doses are used, antisense nucleic acids such as siRNAs and shRNAs still appear to involve the risk of seriously hampering normal cell function, resulting in severe side effects in vivo. Such antisense nucleic acids should then be excluded from further research, even though they may be quite effective in inhibiting a pathogenic microorganism. On the other hand, if a candidate compound appears not to seriously hamper cell function, meaning that cell function is maintained between 85% and 115%, preferably between 90% and 110%, most preferably between 95% and 105% in the presence of said candidate compound, said candidate compound is considered suitable for medicament development.

In one aspect of the invention, therefore, a candidate compound which is capable of inhibiting a pathogenic micro-organism in vitro is introduced into a cell wherein said pathogenic micro-organism, if present, is capable of replicating and/or proliferating. Subsequently, it is determined whether said cell comprising said candidate compound exhibits between 85% and 115%, preferably between 90% and 110%, most preferably between 95% and 105% cell function as compared to the same kind of cell which has not been provided with said candidate compound. If this is the case, said candidate compound is selected for further testing and/or medicament development. If this is not the case, said candidate compound is rejected. Optionally, a candidate compound which seriously hampers cell function and which comprises a nucleic acid sequence of interest is modified such that the same nucleic acid sequence of interest is present in another nucleic acid construct. For instance, impaired cell function can be due to the loop sequence or sense sequence of a shRNA. In such case, the loop sequence is for instance altered and the resulting shRNA is tested again. Alternatively, another kind of nucleic acid construct (such as a miRNA construct) having the same nucleic acid of interest is tested.

In one embodiment, in vivo cell function is determined, for instance by providing a non-human animal such as for instance a mouse or a rat with cells which comprise a candidate compound and, subsequently, assessing cell functioning. Such cells preferably originate from humans. Preferably, a non-human test animal is used which is engrafted with a human hematopoietic system, such as for instance the mouse described in Ter Brake et al, 2009. Such animals are particularly useful for testing the effects of a candidate compound upon human cells.

In a preferred embodiment, however, cell function is (firstly) assessed in an in vitro assay. Such assay is easier and often quicker to perform and does not require test animals.

As used herein, the term “cell function” is defined as at least one function of a cell which it is normally capable of performing in vivo. For instance, cell function encompasses cell metabolism. In one preferred embodiment, the term “cell function” encompasses cell growth, cell replication and/or cell development.

Preferably, cell function is determined of a cell which comprises a candidate compound and which does not comprise said pathogenic micro-organism. This way, the effect of a candidate compound upon the cell is better determined, without interference of said pathogen. In a particularly preferred embodiment, growth, replication, proliferation and/or development of cells comprising a candidate compound is determined and compared to the growth, replication, proliferation and/or development of the same kind of cells which have not been provided with said candidate compound. If cells comprising said candidate compound appear to exhibit between 85% and 115% cell growth and/or proliferation as compared to the same kind of cells which have not been provided with said candidate compound, it is concluded that said candidate compound does not involve too many adverse side effects in vivo and has therefore a higher chance of being suitable for use as a medicament as compared to candidate compounds which involve a reduction of cell growth and/or proliferation by at least 85% or an increase of cell growth and/or proliferation by at least 115%. The last mentioned candidate compounds will be less suitable for use as a medicament, in view of adverse side effects and/or tumorigenicity and will therefore not be selected. Further provided is therefore a method according to the invention, comprising comparing growth and/or proliferation of said cell comprising said candidate compound as compared to the growth and/or proliferation of the same kind of cells which have not been provided with said candidate compound, and determining whether said cell comprising said candidate compound is capable of growing and/or proliferating at a rate of between 85% and 115%, preferably between 90% and 110%, most preferably between 95% and 105% as compared to the same kind of cells which have not been provided with said candidate compound;

wherein, if said cell comprising said candidate compound is capable of growing and/or proliferating at a rate of between 85% and 115%, preferably between 90% and 110%, most preferably between 95% and 105% as compared to the same kind of cells which have not been provided with said candidate compound, said candidate compound is selected as suitable for further testing and/or development of a medicament, and if said cell comprising said candidate compound is capable of growing and/or proliferating at a rate of less than 85% or more than 115%, as compared to the same kind of cells which have not been provided with said candidate compound, said candidate compound is excluded from further development of a medicament.

In another aspect, a candidate compound is introduced into a test cell wherein a pathogenic micro-organism, if present, is capable of replicating and/or proliferating, and said test cell is infected with said pathogenic micro-organism at a dose which would result in replication of said pathogenic micro-organism in the same kind of cells which do not comprise said candidate compound. Said test cell may be infected with said pathogen before said test cell has been provided with said candidate compound, or the test cell may be provided with said candidate compound and with said pathogen simultaneously. Preferably, however, said test cell is infected with said pathogen after said test cell has been provided with said candidate compound. Subsequently, it is determined whether replication and/or proliferation of said pathogenic micro-organism in test cells comprising said candidate compound is inhibited during culturing for at least 80 days. This embodiment is called an “extended culturing assay”. Preferably, it is determined whether replication and/or proliferation of said pathogenic micro-organism in test cells comprising said candidate compound is inhibited during culturing for at least 100 days. In one embodiment, it is determined whether replication and/or proliferation of said pathogenic micro-organism in test cells comprising said candidate compound is inhibited during culturing for about 105 days. If replication and/or proliferation of said pathogenic micro-organism in test cells comprising said candidate compound is inhibited during culturing for at least 80 days, more preferably at least 100 days, it indicates that said candidate compound is suitable for long-term protection. Said candidate compound is then selected as suitable for further development of a medicament. If said candidate compound is not capable of inhibiting replication and/or proliferation of said pathogenic micro-organism during at least 80 days, it is less suitable for further development of a medicament. It has been shown by the present inventors that an extended culturing assay allows for a better selection than current assays, because the HIV inhibition capacity of some candidate compounds only lasts for about 50-70 days. Such candidate compounds are less suitable for therapeutic use.

In this embodiment a dose of pathogen is preferably used which is indicative for natural pathogen loads in infected humans. Preferably, a (viral) dose of less than 2 ng CA-p24 is used. Although known in the art, the term CA-p24 is explained in the Examples. Further provided is therefore a method according to the invention, comprising infecting said cell culture with said pathogenic micro-organism at a dose of less than 2 ng CA-p24 and determining whether replication and/or proliferation of said pathogenic micro-organism in cells comprising said candidate compound is inhibited during culturing for at least 80 days, more preferably for at least 100 days. If replication and/or proliferation of said pathogenic micro-organism in said cells comprising said candidate compound is inhibited during culturing for at least 80 days, more preferably for at least 100 days, it indicates that said candidate compound is suitable for further development of a medicament. Otherwise, said candidate compound is rejected.

In yet another aspect, a candidate compound is introduced into a test cell wherein a pathogenic micro-organism, if present, is capable of replicating and/or proliferating, and said test cell is infected with said pathogenic micro-organism at a dose which firstly results in inhibition of replication of said pathogenic micro-organism during at least 20 days and subsequently results in replication of said pathogenic micro-organism in said cell. Subsequently, it is determined whether at least 80% of the replicating micro-organisms are escape mutants of said pathogenic micro-organism. This embodiment is called an “escalating dose assay”. When high doses of pathogen are used, which doses are often much higher than natural pathogen loads in infected human individuals, some replication will be observed after prolonged culturing of at least 20 days, even though a candidate compound capable of inhibiting said pathogen is present in the cells. In such cases, it is determined whether much wild type pathogen is replicating or whether predominantly escape mutants are present. If the presence of a candidate compound results in the presence of at least 80% escape mutants, it indicates that said candidate compound is particularly well capable of inhibiting wild type pathogen. Such candidate compound is then selected for further testing and/or development of a medicament. If, on the other hand, more than 20% of the replicating pathogens are wild type pathogens, it indicates that said candidate compound is not so well capable of inhibiting wild type pathogen. Such candidate compound is then excluded from medicament development.

In one aspect of the invention, therefore, a candidate compound is introduced into a test cell wherein said pathogenic micro-organism, if present, is capable of replicating and/or proliferating, and the test cell is infected with said pathogenic micro-organism at a dose which firstly results in inhibition of said pathogenic micro-organism during at least 20 days and subsequently results in replication of said pathogenic micro-organism in said cell. To determine which dose of ng CA-p24 has to be added to give such a replication pattern the escalating dose assay can be used as described below. In one preferred embodiment a culture of said cells is infected with said pathogenic micro-organism at a dose of at least 2 ng CA-p24. Subsequently, it is determined whether at least 80% of the replicating micro-organisms are escape mutants of said pathogenic micro-organisms. If this is the case, said candidate compound is selected for further testing and/or development of a medicament.

As said before, said test cell may be infected with said pathogen before said test cell has been provided with said candidate compound, or the test cell may be provided with said candidate compound and with said pathogen simultaneously. Preferably, however, said test cell is infected with said pathogen after said test cell has been provided with said candidate compound.

In an escalating dose assay according to the invention, test cells are infected with a pathogenic micro-organism at a dose which firstly results in inhibition of replication of said pathogenic micro-organism during at least 20 days and subsequently results in replication of said pathogenic micro-organism in said cell. Preferably, several doses of pathogens are used ranging from a dose which results in inhibition of replication up to a dose that causes replication within a two week period. Preferably these doses range from 0.01 ng CA-p24 to 500 ng of CA-p24. This escalating dose assay enables the rapid evaluation of viral inhibition potency of several shRNAs.

In one preferred embodiment, a candidate compound is tested which comprises at least one nucleic acid sequence which is at least 70% complementary to a genomic sequence of said pathogenic micro-organism. Such candidate compounds are often very efficient in inhibiting pathogenic micro-organisms. In one particularly preferred embodiment, a method according to the present invention is used for testing potential anti-HIV therapeutics. As outlined in the Examples, a method according to the invention enables the selection of improved anti-HIV nucleic acid combinations which are preferred over currently known therapeutics. In case of anti-HIV research, a method according to the invention is preferably used for testing candidate compounds which comprise at least one nucleic acid sequence with a length of at least 15 nucleotides which is at least 70% complementary to a genomic sequence of HIV, preferably a conserved genomic region of HIV. More preferably, candidate compounds comprising at least one nucleic acid sequence which is at least 80%, preferably at least 85%, more preferably at least 90% complementary to a genomic sequence of HIV are tested. For testing potential anti-HIV therapeutics, a method according to the present invention is preferably performed with test T-cells, because T-cells are the natural host for HIV.

As said before, it is preferred to test, select and use nucleic acid molecules comprising at least one shRNA and/or at least one siRNA and/or at least one miRNA structure because these kinds of nucleic acids are capable of specifically and efficiently cleaving a target (pathogenic) sequence of interest. Such nucleic acids therefore enable the production of potent and specific medicaments. Further provided is thus a method, a combination of nucleic acid sequences, a use, a pharmaceutical composition, a vector, a gene delivery vehicle or a host cell according to the invention, wherein said at least one nucleic acid sequence or said combination of nucleic acid sequences comprises at least one shRNA and/or at least one siRNA and/or at least one miRNA structure.

A method according to the present invention is particularly suitable for testing two or more candidate compounds (herein called a plurality of candidate compounds).

Each candidate compound is subjected to at least one selection method according to the invention, either separately or together with at least one other candidate compound, and the test results of each candidate compounds are compared with each other. Preferably, said plurality of candidate compounds is subjected to at least two different test methods according to the invention. More preferably, said plurality of candidate compounds is subjected to at least three different test methods according to the invention in order to test both long term inhibition and the risk of adverse side effects. Subsequently, at least two candidate compounds are preferably selected which are suitable for further testing and/or development of a medicament. Preferably, at least three candidate compounds are selected. In a particularly preferred embodiment, at least four candidate compounds are selected. Of course, it is preferred to select candidate compounds which have satisfactory test results in all tests performed. Said at least two selected candidate compounds are subsequently used for further research and/or therapy development. One embodiment thus provides a method for selecting at least two compounds from a plurality of candidate compounds comprising at least two nucleic acid sequences which are complementary to a genomic sequence of a pathogenic micro-organism and which are capable of inhibiting a pathogenic micro-organism, the method comprising subjecting said plurality of candidate compounds to at least one, preferably at least two, more preferably at least three different test methods according to the invention, and selecting from said plurality of candidate compounds at least two, preferably at least three, more preferably at least four compounds which are suitable for further testing and/or development of a medicament.

The invention is further illustrated by the following examples. These examples are not limiting the invention in any way, but merely serve to clarify the invention.

### BRIEF DESCRIPTION OF THE DRAWINGS

FIG. 1: Anti-HIV-1 shRNA inhibitors. A) HIV-1 genome showing the target sequences for the shRNAs used in this study. L=Leader, G=Gag, P=Polymerase, PV=Pol/Vif, RT=Rev-Tat, N=Nef. Several regions were targeted by multiple overlapping shRNAs, e.g. L2-L9. B) The third generation lentiviral vector JS1 was used with the shRNA transcribed from the H1 polymerase III promoter.

FIG. 2: Transient cotransfection assays. A) Transient luciferase reporter assay. For each group of shRNAs that target a specific region another luciferase reporter was constructed and used. JS1 was always used as a control and this luciferase activity was set at 100%. B) Transient cotransfection with the HIV-1 molecular clone. The empty lentiviral vector JS1 was used as a control. All cotransfections included renilla luciferase, which was used to correct for variation in the transfection efficiency.

FIG. 3: Extended culturing of HIV-1 infected stable anti-HIV-1 shRNA T-cells. For a period of 106 days we scored syncytia formation of HIV-1 infected stable anti-HIV-1 shRNAs. For each shRNA cell type, 6 cultures were followed until syncytia formation and cell death occurred. In this graph, we depicted the day when full blown syncytia and massive cell death was scored (open circle). Other cultures were maintained up to day 106 (closed circles). The bars indicate the average time needed for full blown syncytia and cell death to occur, if no culture escaped this was set at 106 days.

FIG. 4: HIV-1 escape and the U5-AUG interaction.

A) Shown is the LTR-gag part of the HIV-1 DNA and the targets for shRNA L2-L9. The indicated U5 and AUG domains of the HIV-1 RNA genome can interact by base pairing. The AUG start codon is marked with an asterisk.
B) Acquired mutations in the AUG domain are indicated. The shRNA culture is indicated on the left, the number of occurrences on the right. C) The proposed U5-AUG basepairing interaction and the acquired mutations.

FIG. 5: HIV-1 dose escalation assay. A selection of each group of shRNAs was tested on stable cell lines with a single copy of shRNA lentivirus per cell on the ability to suppress different viral loads of HIV-1. Light grey, dark grey and black bars represent the modest, intermediate and potent inhibitors, respectively. Syncytia was scored from no syncytia formation to full blown syncytia formation. In this graph we depicted how much HIV-1 (ng/ml CA-p24) was needed to get massive syncytia formation within 14 days.

FIG. 6: Measuring the effect of shRNAs in PM1 T cells cultures.

PM1 cells were transduced with an MOI of approximately 0.15, most GFP+ cells will only have one integration of shRNA expressing or control lentivirus per cell, after sorting almost all selected PM1 cells are GFP+, but a few GFP− cells remain. When the shRNA has a negative effect on cell growth this can result in outgrowth of the GFP− cells that not containing a lentiviral vector. Thus after a period of the culturing these sorted cells were analyzed on the % of GFP+ cells by FACS analysis. For shRNA L9 and G5 a reduction of GFP+ cells is observed, compared with the control and the other shRNAs. This is indicative of a disturbance by these shRNAs on normal cell growth.

FIG. 7: Measuring the effect of shRNAs in SupT1 cells cultures.

SupT1 cells were transduced with high concentrations of lentiviral vectors. Over time GFP+ was measured. For the shRNA G5 a reduction in GFP+ cells was observed, indicating a potential disturbance of the shG5 on normal cell growth.

FIG. 8: RNAi induced by different RNA structures

FIG. 9: Schematic overview of RNA interference with shRNA

FIG. 10: Schematic overview of gene regulation by miRNA

Non-limiting, schematic overview of the natural microRNA pathway. Polymerase II or III produces the primary transcript, pri-miRNA, that encodes miRNA. Many miRNAs are clustered and transcribed as polycistronic transcripts. This pri-miRNA is processed into a pre-miRNA with a 5′ monophospate and a 3′ 2 nt hydroxyl overhang by a microprocessor containing the RNase III enzyme Drosha and a dsRNA binding protein termed DGCR8/Pasha. MicroRNAs present in introns (mirtrons) are also processed into pre-miRNAs through a distinct splicing route as depicted in the top at the right side in this Figure. The pre-miRNA is made in the nucleus and exported to the cytoplasm by Exportin-5. In the cytoplasm the RNAse III endonuclease Dicer binds to the pre-miRNA and cleaves the base-paired stem approximately 22-nt away from its base, generating a 2-nt overhang at the 3′ end. The RNA-induced silencing complex (RISC) with as main proteins Dicer and Argonaute 2 (Agog) elicits unwinding of the miRNA and loading of one strand (guide strand) in the complex, the other strand gets degraded (passenger strand). Preferably the RNA strand with the lowest thermostability at its 5′ duplex end gets incorporated into the complex. In mammals, silencing is mainly elicited by translational repression of the targeted mRNA. The most important determinant for mRNA cleavage or translational repression is target RNA recognition based on perfect or near-perfect complementarity of the 5′ “seed” region of the miRNA with the mRNA, respectively. Typically RISC binds to the target-seed sequences in the 3′ untranslated region (3′UTR) of the mRNA. The number of target-seed sequence in the 3′UTR and the distance between them plays a role in the silencing efficiency. Mammalian miRNA base pairing occurs mainly by imperfect complementarity with the mRNA and only a few cases of complete complementarity and cleavage are known in humans. Endonucleolytic cleavage of the targeted mRNA occurs opposite of the 10^{th} and 11^{th} nucleotide of the miRNA. The cleaved mRNA is subsequently degraded.

It is a rare event that natural miRNAs elicit cleavage in mammalian cells, however artificial dsRNAs with full complementarity to an mRNA can direct target cleavage. Artificial dsRNA can be expressed by several methods including: mature siRNAs, short hairpin RNAs or artificial microRNAs. Thus the natural miRNA pathway can be used for therapeutic elective intracellular down regulation of a specific mRNA. This therapeutic approach is interesting for targeting RNAs of invading organism such as HIV-1.

FIG. 11: Non-limiting example of HIV therapy wherein T-cells, progenitor cells and/or stem cells of an HIV patient are provided ex vivo with nucleic acids according to the invention.

FIG. 12: Non-limiting examples of expression cassettes comprising nucleic acids according to the invention.

### EXAMPLES

AIDS remains a serious problem with millions of newly HIV-1 infected individuals each year. Antiviral therapy that prevents or delays the onset of AIDS has improved the quality of life and life expectancy. However, drug resistance and severe side effects remain a serious problem. These issues argue for a continued search for new antiviral therapeutic strategies. RNAi is a promising antiviral strategy for HIV-1 and other pathogenic viruses. RNAi therapeutics rely on the usage of the endogenous host cell mechanism for post-transcriptional gene silencing in which double-stranded RNA triggers the sequence-specific cleavage of a fully complementary mRNA. Because chronic infections like HIV-1 require a durable gene therapy, we and others used intracellularly expressed anti-HIV shRNAs to inhibit virus replication. Processing of the shRNA by Dicer into the active siRNA form is followed by loading into the RNA-induced silencing complex (RISC) and mRNA inactivation.

All single drug HIV-1 therapies are susceptible to viral escape, including a mono-shRNA therapy. This can be counteracted by the combination of several highly potent shRNAs. Previously, we performed a large screen to identify effective shRNAs that target highly conserved regions of the HIV-1 RNA genome (Ter Brake et al, 2006). The candidate shRNA inhibitors were selected based on at least 65% reduction of virus production in a transient cotransfection with the HIV-1 plasmid pLAI and 75% target coverage among the more than 600 HIV-1 isolates present in the Los Alamos National Laboratory database (http://www.hiv.lanl.gov/). This screen yielded 20 candidate shRNAs that could potentially be used for a combinatorial RNAi approach.

To document the optimal way to test such antiviral compounds, in this Example we screened these candidate shRNA inhibitors in different assays ranging from transient transfection to long-term virus infections. First, we performed transient transfection experiments with the shRNA expressing vector and either the matching luciferase reporter or the HIV-1 molecular clone. Second, we generated stable shRNA-expressing T cell lines that were challenged with HIV-1 and followed for an extended period to monitor viral escape. This culture method is certainly most laborious and time consuming, but also optimally suited for shRNA selection. Third, we performed short term infection experiments with these cells and escalating doses of virus. We compared the outcome of these assays and found improved anti-HIV shRNA combinations.

### Materials and Methods

### Plasmid Construction

Construction of the JS1 lentiviral vector, luciferase target pGL-3 reporters and the shRNA sequences have previously been described (Ter Brake et al, 2006). The shRNA lentiviral vectors were generated by digestion of the pSUPER constructs (Ter Brake et al, 2006) with XhoI and PstI. The fragment was inserted into the corresponding sites of JS1 (Ter Brake et al, 2006), resulting in JS1-shRNA.

### Cell Culture

Human embryonic kidney 293T cells were cultured as monolayer in DMEM (Gibco BRL) and the human T cell line SupT1 was cultured as suspension cells in advanced RPMI (Gibco BRL), DMEM was supplemented with 10% Fetal Calf Serum (FCS), advanced RPMI with 1% FCS and L-glutamate. Both media contained penicillin (100 U/m) and streptomycin (100 μg/ml) and cell lines were kept in a humidified chamber at 37° C. and 5% CO₂.

### Lentiviral Vector Production and Transduction

Lentiviral vector production, transduction and generation of stable cell lines has been described previously (Ter Brake et al, 2006). SupT1 cells were transduced with a multiplicity of infection (MOI) of 0.15 and GFP-positive cells were selected with live FACS sorting.

### Transient Cotransfection Assays

Cotransfections were performed in the 24-well format, 150.000 293T cells were seeded in 0.5 ml DMEM with 10% FCS without antibiotics. Transfection was performed the next day when the cells reached a confluence of approximately 70%. DNA lipofectamine mix was prepared according to the manufacturer's protocol (Invitrogen) and added to the cells. The luciferase reporter and JS1-shRNA vector were cotransfected in a 1:1 ratio of 100 ng per well and 2.5 ng renilla expression plasmid (pRL) was included as a transfection control. Cells were lysed twenty-four hours post transfection and a dual luciferase assay (Promega) was performed according to the manufacturers protocol, but the reaction volume was downscaled to 25 μl. We also cotransfected 100 ng pLAI with 100 ng of JS1-shRNA vector, and 2.5 ng of pRL. The culture supernatant was collected at forty-eight hours after transfection and supernatant was used to measure CA-p24 by ELISA as a measure of virus production and cells were lysed for renilla measurement. To correct for transfection variation, the CA-p24 values were divided by the Renilla values. Transfections were performed at least twice and in duplicate.

### Virus Dosage Expressed in ng CA-p24

The ng CA-p24 is the translation for the amount of virus we add to our testing cultures with a standard number of cells. We produce the HIV by transfecting the viral plasmid pLAI using lipofectamin2000 in 293T cells according to the manufacturer's lipofectamin protocol. We collect cell supernatant that contains the viral particles. We spin down the cell debris and aliquot the virus samples. A CA-p24 ELISA is performed measuring the concentration of CA-p24 protein, in ng CA-p24/ml, from the viral capsid. Subsequently we perform a titration with the produced virus by using several dilutions of the produced virus on a fixed number of SupT1 cells in a fixed volume, to determine the minimal amount of virus required to obtain a good infection on SupT1 cells: the peak of infection has to be reached in these unprotected SupT1 cells between day 7 and 14, where the virus concentration measured by CA-p24 usually is between 100-1000 ng/ml. The virus amount used in subsequent infection experiments is expressed in ng CA-p24. Routinely we infect 5 ml unprotected SupT1 cell cultures containing 5 millions cells with about 1 ng CA-p24 to obtain peak infection between day 7 and 14.

Long Term Culturing of SupT1-shRNA Cells Upon HIV-1 Challenge

Each SupT1-shRNA cell line (3 ml cultures, 1.5×10⁶ cells) was infected in 6-fold with a fixed amount of HIV-1. As control cells, the SupT1 cells transduced with the empty JS1 vector or the inactive shRNA PV4 were used. Virus replication was monitored by HIV-induced cellular syncytia formation and cell-free virus was passaged to SupT1-shRNA cells and syncytia was documented to monitor virus replication.

### Analysis of Escape Virus Variants

Cellular DNA of re-infected cells was isolated as previously described (Konstantinova et al, 2006). Integrated proviral DNA sequences were PCR amplified and sequenced. We used several PCR primers and their 5′ end start position in the LAI sequence is indicated between brackets. Leader (for sequencing L3, L5-8), sense CACACACAAGGCTACTTCCCTGATTAGCAGAACT (104) and antisense ACCTTGCTGCAGCCTCTATCTTGTCTAAAGCTTCCTTGGTG (1125). Pol (for sequencing P9, P29, P44, P45) sense GAAGCAGAAGTTATCCCAGCAGAGACAGGGC (4567) and antisense CCCAAGCTTCTAATCCTCATCCTGTCTACTTGCC (5157) Tat-Rev (for sequencing RT4) sense ACCTTGTCTAGAATGGAGCCAGTAGATCCTAGACTAGAGCCCTG (4567) and antisense GAAGCAGAAGTTATCCCAGCAGAGACAGGGC (6407). PCR products were cloned into the pCR2.1 TOPO vector and bacterial clones were subsequently PCR amplified and sequenced with the T7 and/or M13R primer compatible with the pCR2.1 TOPO vector. True genotypic virus escape was defined as at least two identical clonal mutated target sites sequences for a culture.

HIV-1 Escalation Infection of SupT1-shRNA Cells

The shRNA-SupT1 cells were seeded in a 48-well plate (200,000 cells/well). Different amounts of HIV-1 were added (CA-p24 end concentration range from 0.1 to 500 ng/ml). The cultures were inspected for syncytia formation 3, 7, 10 and 14 days post infection. Cells were split at day 7 and the culture was maintained for another week.

### Results

The Set of Active Anti-HIV-1 shRNAs

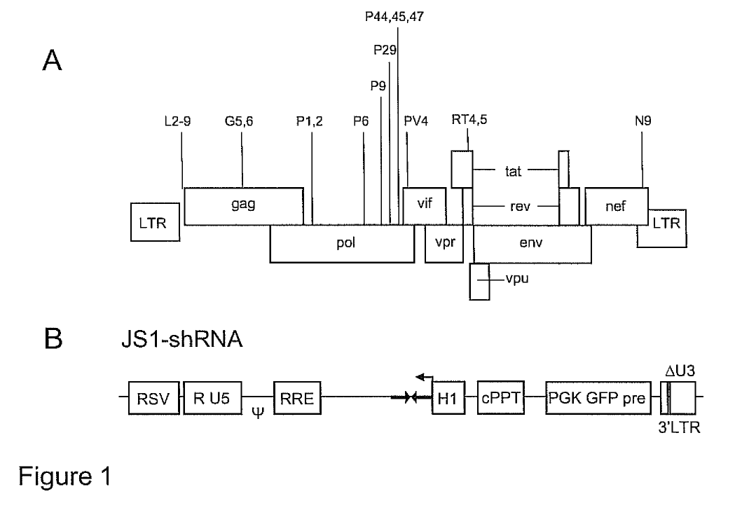

A large scale screen of 86 shRNAs targeting highly conserved HIV-1 regions was previously performed (Ter Brake et al, 2006). This screen yielded 20 shRNA candidates for which the targets in the untranslated leader (L), gag gene (G), pol gene (P) and the overlapping tat-rev genes (RT) are indicated on the HIV-1 genome in FIG. 1A. Some regions were targeted by multiple shRNAs that were tiled, moving one nucleotide per shRNA (e.g. L2-9). The shRNAs were cloned as H1-driven polymerase III expression cassette in the lentiviral vector JS1 (FIG. 1B). The lentiviral GFP-marker was used to select transduced cells of the SupT1 T cell line. We also prepared control cells transduced with the empty lentiviral vector JS1, and cells expressing an inactive shRNA that targets the pol-vif overlap region (PV4). As positive control, we used a shRNA targeting the Nef region (N9). This potent N9 inhibitor does not target a highly conserved HIV-1 region and may therefore provide more possibilities for viral escape. These 23 lentiviral vectors (22 shRNA-encoding and the empty JS1 vector) were used in the transient assays and for the generation of stable T cell lines.

Transient Transfection Assays to Score shRNA Antiviral Activity

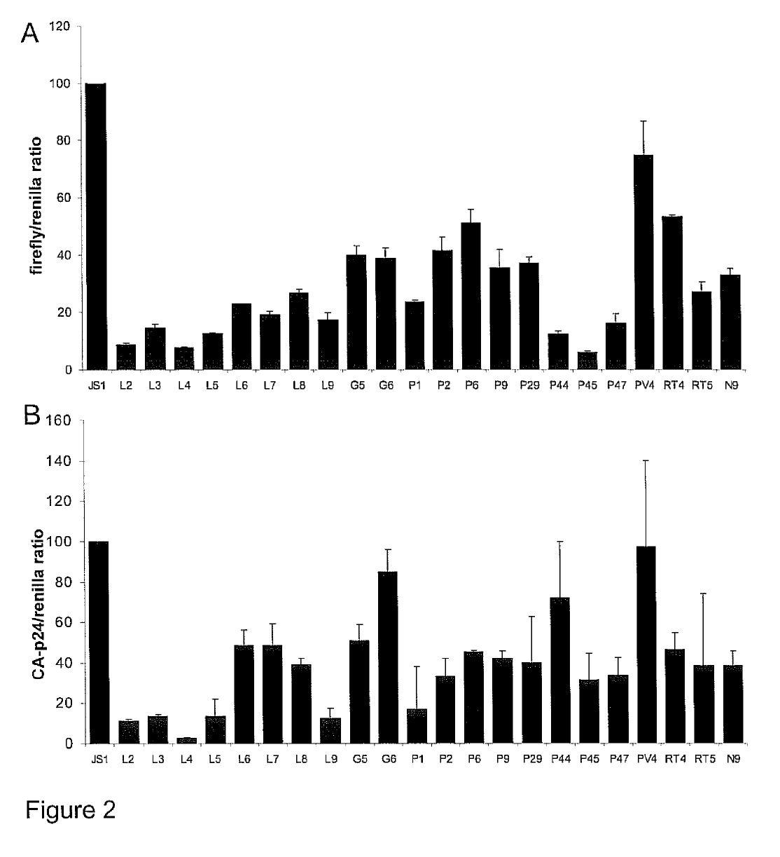

To evaluate if transient co-transfection assays can identify the most potent shRNAs, two transient inhibition assays were performed with either a luciferase reporter or the HIV-1 molecular clone. We first co-transfected 293T cells with each of the 22 different JS1-shRNA plasmids and the corresponding firefly luciferase reporter constructs with the target sequence cloned in the 3′UTR. As negative control we used the JS1 empty vector. A fixed amount of renilla luciferase plasmid was included to control for the transfection efficiency and the firefly/renilla ratio was calculated. Values obtained with JS1 and the respective luciferase reporter was set at 100% for each reporter (FIG. 2A). As expected, we measured relatively efficient inhibition, ranging approximately from 50% to more than 90%, except for the inactive shRNA PV4.

We next cotransfected the HIV-1 plasmid pLAI with the JS1-shRNA set. The HIV-1 RNA genome contains targets for all shRNAs and the empty vector JS1 served again as negative control. Virus production was measured by CA-p24 production in the culture supernatant, divided by the renilla values and related to the JS1 control that was set at 100% (FIG. 2B). Again a potent knockdown is observed for most shRNAs except for the inactive shRNA PV4. The results of the two transient assays do overlap considerably, e.g. showing that shRNA L4 is among the most potent inhibitors.

Extended Culturing of HIV-1 Infected shRNA Cells

We infected the stably transduced shRNA-expressing T cell lines with HIV-1 to test the durability of viral inhibition, an alternative assay that resembles the ultimate gene therapy setting. Six individual SupT1-shRNA cultures were infected per shRNA and the cells were split when needed. Viral replication was monitored over time by scoring syncytia formation. If syncytia were observed, the cell-free viral supernatant was collected to allow reinfection of fresh shRNA cells.

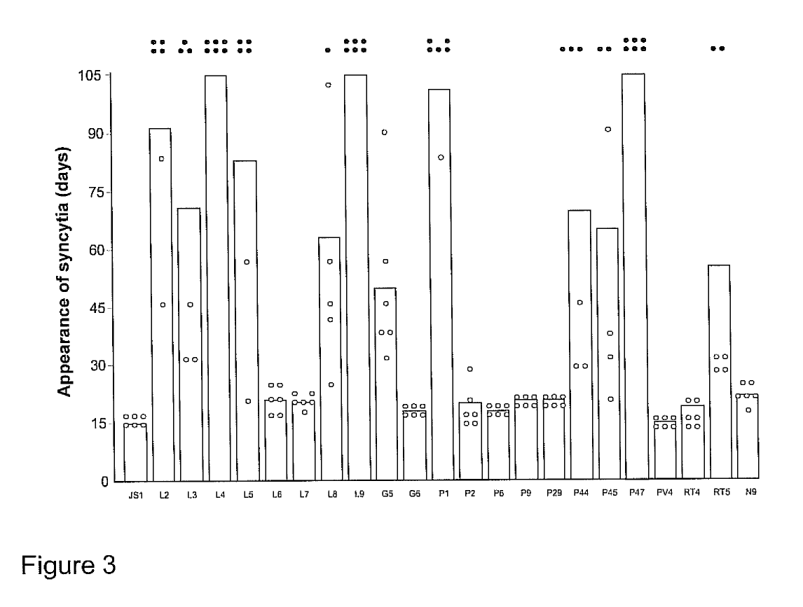

FIG. 3 summarizes the results of the extended culturing assay. The control cell lines (empty vector JS1 and inactive shRNA PV4) supported active virus replication within the first week and all six cell cultures were completely wasted due to massive syncytia after 2 weeks. A group of relative weak shRNA inhibitors (L6, L7, G6, P2, P6, P9, P29, RT4, N9) allowed virus replication to become visible within two weeks and the cells were dead after three weeks. The remaining cultures either occasionally yielded a virus-positive culture at later time points or did not show any sign of virus replication during the 106 day test period. These shRNAs were labeled as the strong and durable inhibitors, respectively (L2, L3, L5, L8, G5, P44, P45, RT5) and (L4, L9, P1, P47). Among the durable inhibitors, only a single P1 culture showed signs of HIV-1 replication around day 50 (indicated as open circle), the other 23 cultures in this group remained virus-free for the test period of 106 days (indicated as closed circles). The delayed virus appearance and its stochastic nature are indicative of viral escape, which is driven by randomly acquired mutations. To assure that we did not miss a low level of ongoing virus replication with the syncytia screen, we also measured CA-p24 levels in the supernatant at the end of the experiment, which confirmed the absence of virus replication (data not shown).

Virus replication as observed for the weak and strong shRNA inhibitor groups can be due to incomplete virus inhibition, such that the wild-type virus can slowly accumulate, also called pseudo-escape. Alternatively, it may indicate the selection of escape variants with a mutation in the target sequence, which is in fact an indication of strong initial inhibition by the shRNA. Thus, it is important to discriminate between these two possibilities. To do so, the replicating viruses were passaged onto the corresponding SupT1-shRNA cells, and subsequent replication is an obvious sign of phenotypic viral escape. We also analyzed the HIV-1 target sequence of possibly evolved virus variants to demonstrate genotypic escape.

For the weak shRNA group, pseudo-escape was apparent in 24 of the 26 cultures that were analyzed. This result confirms the sub-optimal inhibitory activity of these shRNAs. Wild-type sequences that are indicative of partial virus inhibition and pseudo-escape were also observed for a L2, two L3, and a P44 culture. In contrast, true genotypic virus escape was evident in 11 of the 15 cultures in the group of strong shRNA inhibitors.

### Highly Restricted Escape Pattern for the HIV-1 Leader

We observed some interesting patterns among the escape variants. Escape occurs generally by point mutations in the target. We observed a mutational bias towards G-to-A changes and the RNAi-induced virus variants resemble the natural sequence variation among HIV-1 isolates. These evolution characteristics are consistent with our previous RNAi-escape studies.

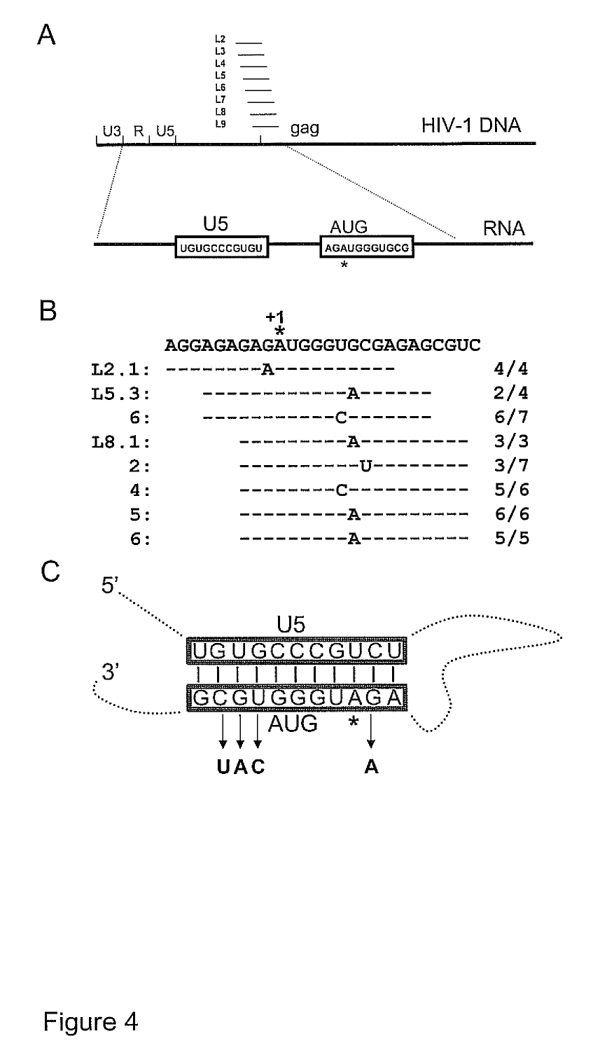

The untranslated leader region of the HIV-1 RNA genome contains conserved regions that encode important biological functions. We tested 8 shRNAs, L2-9, with overlapping targets that cover the gag initiation codon (FIG. 4A). This sequence is also part of the U5-AUG base pairing interaction that is critical for HIV-1 replication (FIG. 4A). Among the leader set, we scored weak inhibition (L6, L7), strong inhibition (L2, L3, L5, L8) with occasional virus escape and durable inhibition (L4, L9). For eleven of the strong shRNA inhibitor cultures that eventually showed virus replication, clonal sequences were analyzed. Eight cultures demonstrated genotypic escape, with mutations present in the target sequence for two or more clones per culture (FIG. 4B). All four mutations observed in the RNAi-escape analysis also represent the most variable positions in natural HIV-1 isolates (data not shown). Interestingly, different shRNAs yielded the same escape mutant. The G-to-A change at position +7 relative to the Gag AUG start codon appears the most favorite escape route, which was selected in 4 cultures (1×L5, 3×L8). The U-to-C change at position +6 was selected twice (1×L5, 1×L8). The U+6C and C+8U are silent in the Gag open reading frame, but the G+7A is not. However, the encoded amino acid substitution from an aliphatic to an aliphatic hydroxyl group (alanine to threonine, 3AT) is also seen in natural HIV-1 isolates.

Most notably, the escape mutations do maintain the U5-AUG duplex (FIG. 4C). In fact, the dominantly observed changes do substitute relative weak G-U base pairs by stronger ones. The G+7A and U+6C mutations result respectively in an A-U and G-C base pair in the U5-AUG duplex structure. We also observed a minor weakening, C+8U, where the base pair G-C was mutated in G-U pairing. There is only one exception in which the base pairing is destroyed (G-1A), the C-G becomes a C A mismatch. The gag initiation codon is never affected, which is in concordance with the fact that the gag protein is essential for viral replication.

### A Transient Dose-Escalating HIV-1 Infection Assay

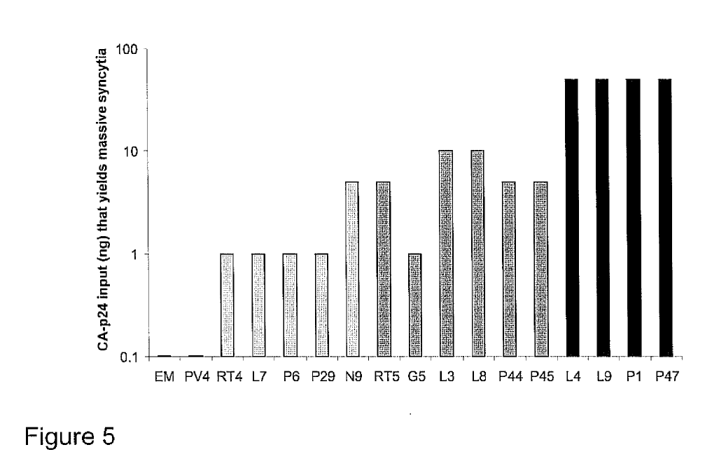

We next tested if there may be a faster method than long-term culturing to identify durable shRNA inhibitors. We made a selection of shRNAs from each potency group and performed short term 14 day infections of the SupT1 T cell lines with escalating HIV-1 doses ranging from 0.1 to 500 ng CA-p24/ml. Cultures were scored for syncytia formation and we plotted the amount of HIV-1 input needed to induce massive syncytia formation within 14 days (FIG. 5). A clear distinction between the three different shRNA groups was apparent. For the durable shRNA inhibitors an input of 50 ng/ml of CA-p24 was required to obtain a full blown infection within 2 weeks. In contrast, the control, weak and strong inhibitor groups required concentrations of respectively 0.1 ng, 1 ng and 1-10 ng CA-p24/ml. This result also indicates that HIV-1 replication can be forced in the presence of any shRNA, but the amount of virus input required is indicative of the potency of the inhibitors. This escalating dose assay was able to identify the four potent and most durable shRNAs.

### Discussion

We compared transient transfection, long-term infection and virus escalation assays for their ability to discriminate between antiviral shRNAs that provide weak, strong or durable HIV-1 inhibition. The two transient co-transfection assays with a reporter construct or HIV-1 molecular clone were able to identify weak inhibitors but did not provide sufficient discrimination between strong and durable shRNAs. Long-term infection of HIV-1 on shRNA-expressing cells, followed by sequencing of putative escape variants, did clearly distinguish between the weak, strong and durable inhibitors. The weak shRNA inhibitors allowed the delayed appearance of the wild-type virus (pseudo-escape due to partial virus inhibition). The strong shRNA inhibitors required the acquisition of a mutation in the target sequence to allow virus breakthrough (true escape). The durable shRNA inhibitors did not allow virus escape.

We thus consider long-term culturing the most optimal way to determine which shRNAs represent durable inhibitors, but the viral escalation assay provides a reasonable alternative. This transient assay was able to identify the same set of durable inhibitors.

Combining all these results, we have devised a strategy for nucleic acid selection. Initial screening could be performed in the transient HIV-1 DNA transfection assay, yielding candidate inhibitors that provide 65% inhibition. Additionally, a dose escalation assay with these candidates is preferably performed to divide the inhibitors in different potency groups. In another experiment, the most effective HIV-1 inhibitors are preferably validated in an extended culturing assay to document possibilities for viral escape.

For a clinical gene therapy we have proposed the use of a lentiviral vector that expresses four anti-HIV-1 shRNAs (Ter Brake et al, 2008). This set includes P1 and P47 from the group of durable inhibitors, but also G5 and RT5 that we now define as strong, but not durable inhibitors. However, RT5 remains an interesting inhibitor that targets the overlapping tat and rev genes, which may restrict viral escape options. It is preferred to replace the relative weak G5 shRNA with the durable inhibitor L4 and/or L9 that were identified in this study.

For in vivo experiments we propose the humanized mouse model (Ter Brake et al, 2009). The great advantage of this model is that human haematopoietic stem cells are used that we also propose to use for the ex vivo gene therapy. These stem cells can develop into mature immune cells that sustain HIV-1 infection. Previously, we demonstrated successful transduction of a shRNA-expressing lentivirus vector in human hematopoietic stem cells (Ter Brake et al, 2009). After introduction of these cells in the mouse, mature human T cells developed. Ex vivo, we demonstrated HIV-1 inhibition in these shRNA expressing cell population.

### Example 2

### A Sensitive Co-Culture Assay to Detect Cell Toxicity

### Introduction

While the aim of a therapeutic drug is the treatment of a disease, often iatrogenic effects are also present. When screening for a therapeutic compound, it is also important to evaluate the risk of these iatrogenic effects.

By testing candidate shRNAs in an early stage unsuitable shRNAs will be removed from further screening. This prevents the unnecessary screening of these hairpins that would have most likely resulted in a costly loss of materials, test animals, time and money. To evaluate the candidate shRNAs we developed in vitro assays in which we screen for adverse effects of our shRNAs on the cells by comparing their replication capabilities with that of non-treated cells.

Treated and non-treated cells were mixed to create defined cocultures. The lentiviral treated cells contain GFP as a marker and can thus be distinguished from the non-treated cells by simple FACS analysis. Since these cells are in the same culture they are treated equally and a growth advantage or disadvantage of the treated cells can easily be scored. When the percentage of treated cells decreases this indicated that there is a growth disadvantage or cell death. When the number of treated cells increases, it might be an indication that the shRNA elicits a tumorigenic effect.

### Materials and Methods

### Plasmid Construction

Construction of the JS1 lentiviral vector is previously described (Ter Brake et al, 2006). The shRNA lentiviral vectors were generated by digestion of the pSUPER constructs (Ter Brake et al, 2006) with XhoI and PstI. The fragment was inserted into the corresponding sites of JS1 (Ter Brake et al, 2006), resulting in JS1-shRNA.

### Cell Culture

The human T cell lines SupT1 and PM1 were cultured as suspension cells in advanced RPMI (Gibco BRL) supplemented with 1% FCS and L-glutamate, penicillin (100 U/m) and streptomycin (100 μg/ml) and cell lines were kept in a humidified chamber at 37° C. and 5% CO₂.

### Lentiviral Vector Production and Transduction

Lentiviral vector production, transduction and generation of stable cell lines has been described previously (Ter Brake et al, 2006). PM1 cells were transduced with a multiplicity of infection (MOI) of 0.15 and GFP-positive cells were selected with live FACS sorting. SupT1 cells were transduced with lentiviral vectors resulting in 70 to 85% GFP positive cells. Cultures were analyzed by FACS analysis at several time points.

### Results

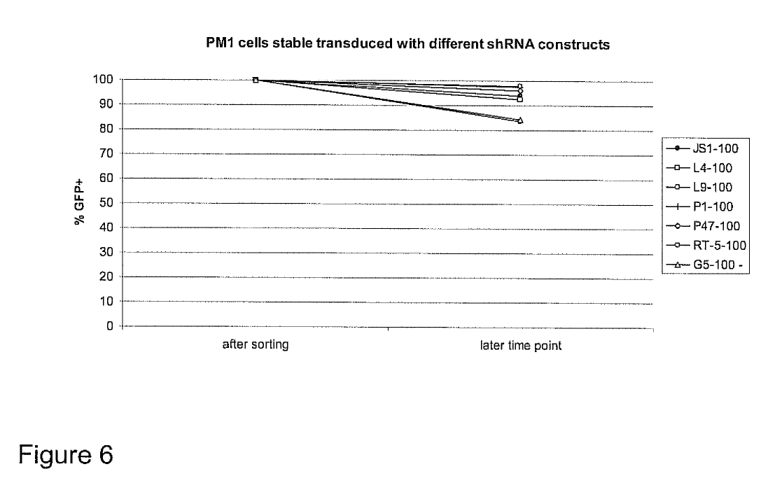

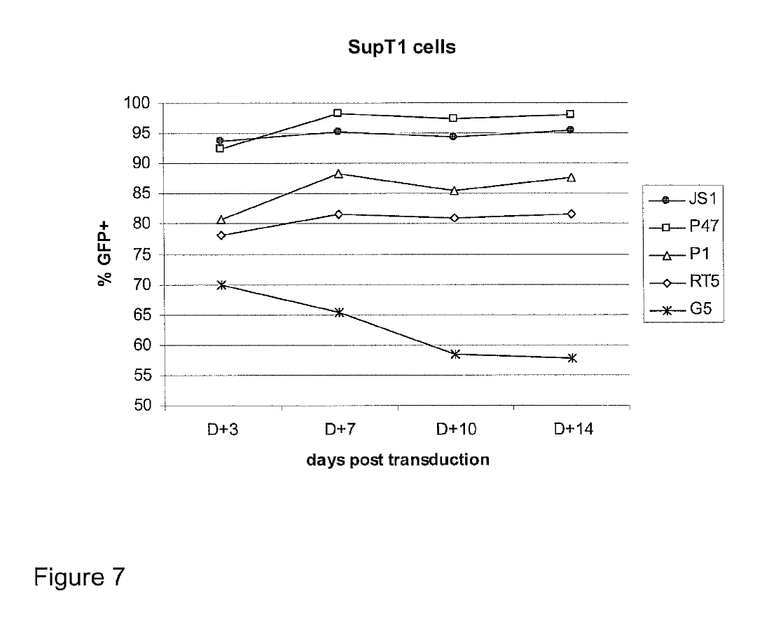

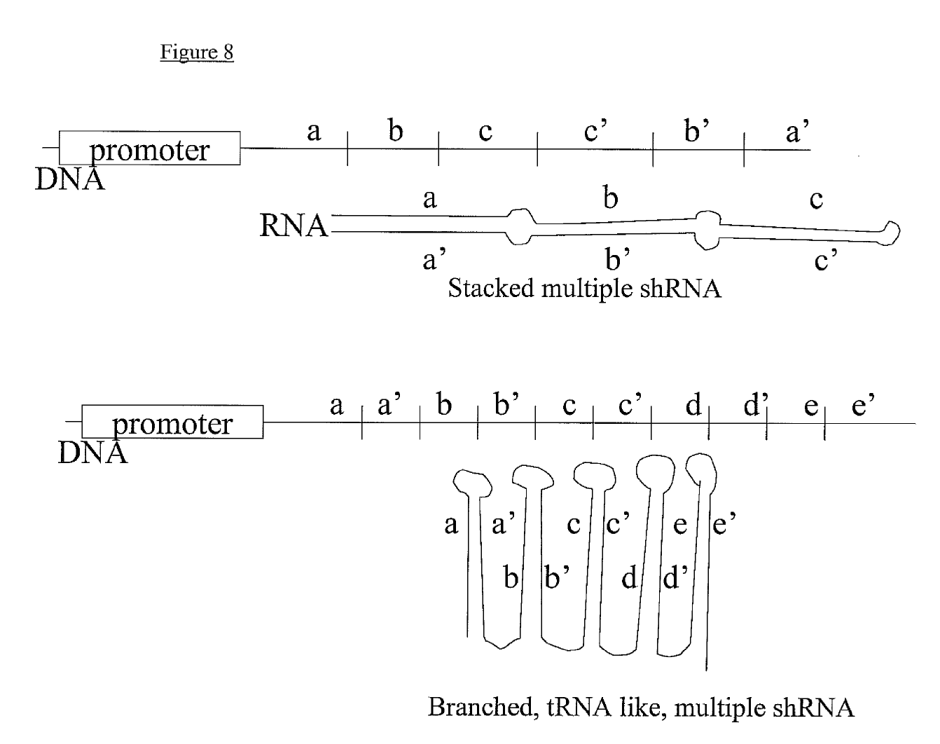

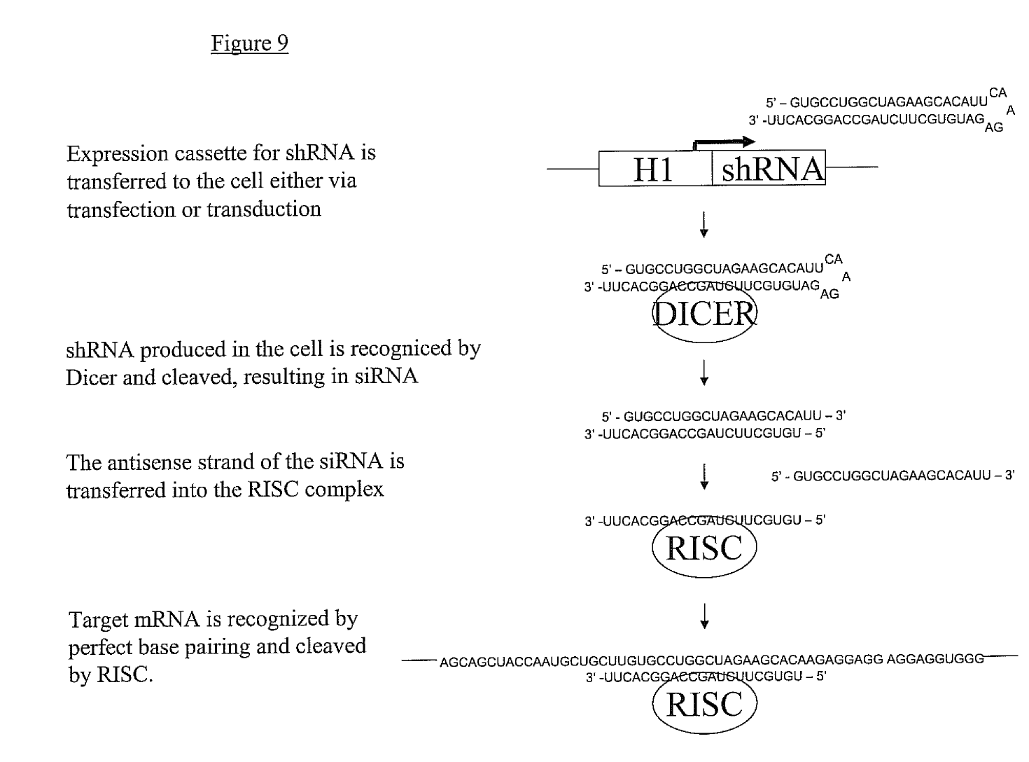

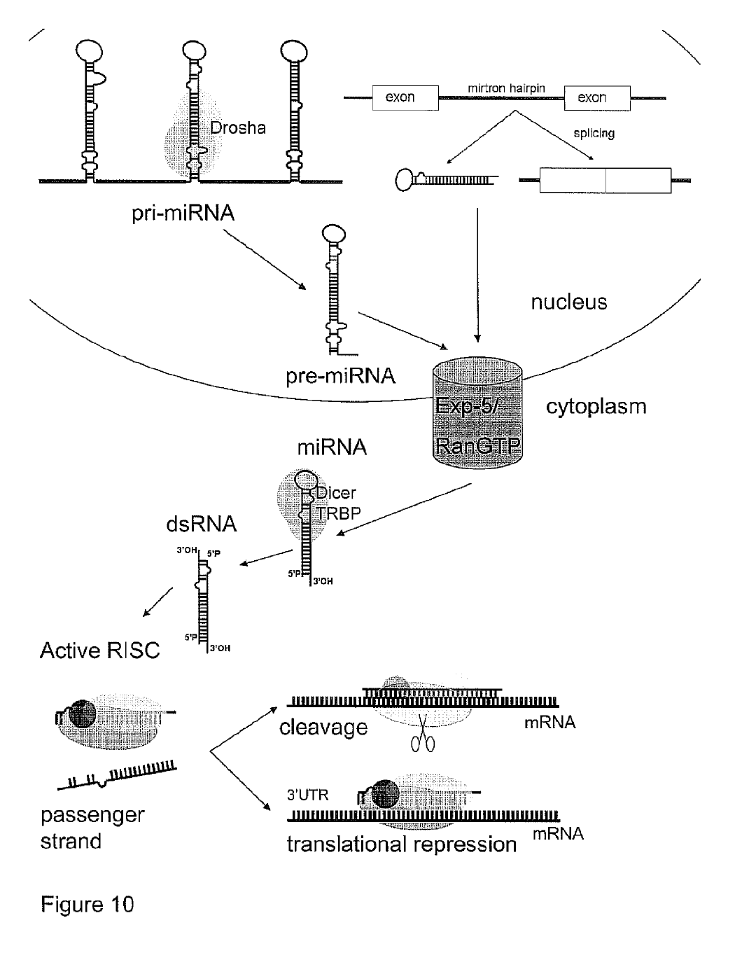

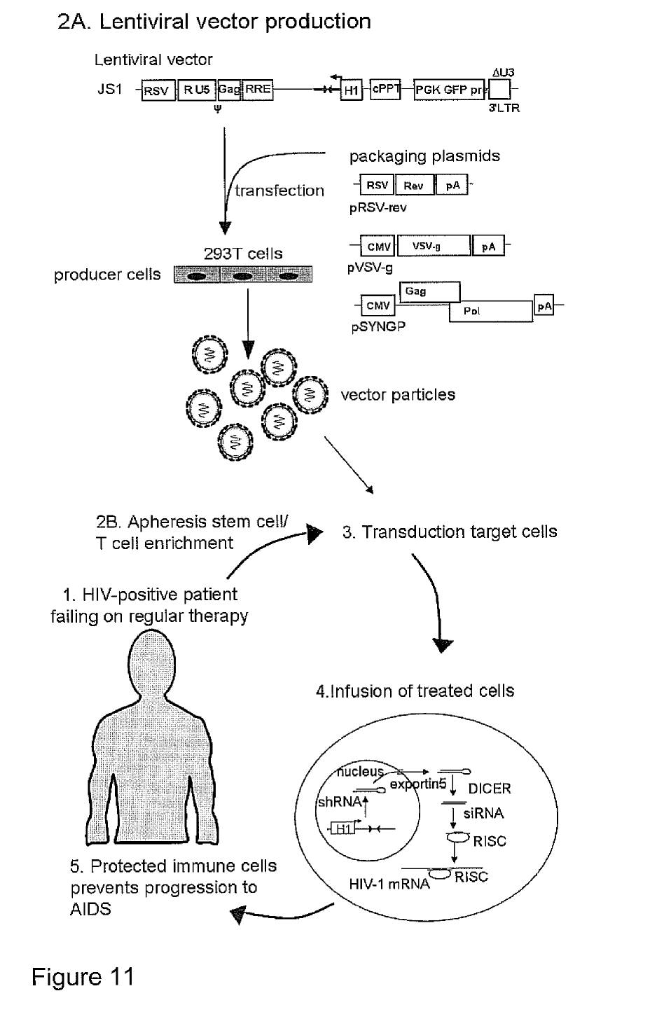

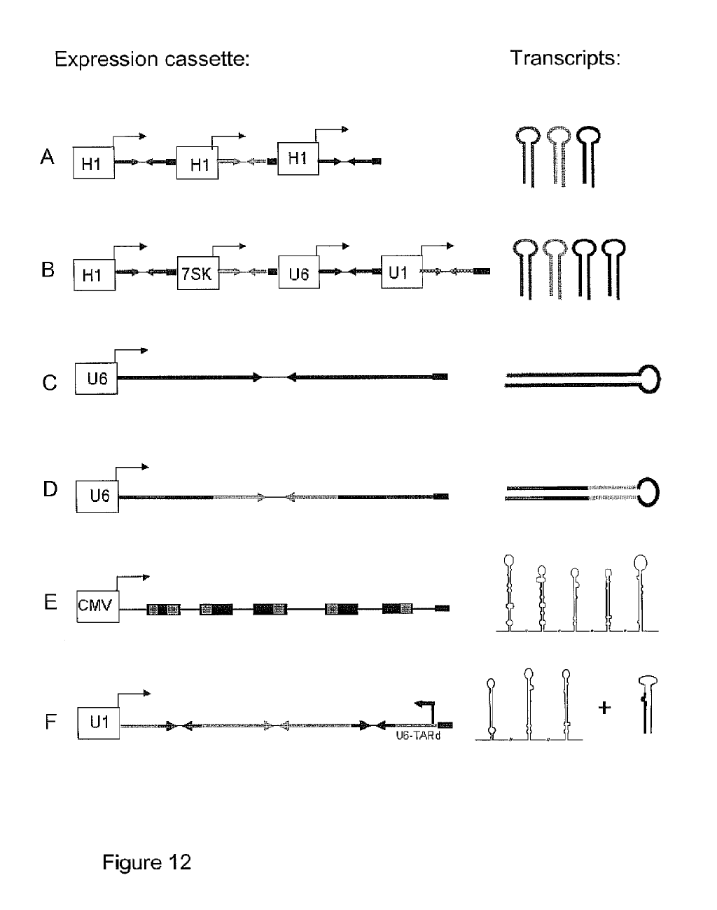

PM1 cells were transduced with an MOI of approximately 0.15 such that most GFP+ cells will have one integrated shRNA expressing cassette or control lentivirus. After sorting for GFP positivity most PM1 cells are GFP+, but a small percentage GFP negative cells remain. When shRNA expression has a negative effect on cell growth this can result in outgrowth of this small population of GFP negative cells. Thus, after a variable period of culturing we analyzed the % of GFP positive cells by FACS analysis. For shRNA L9 and G5, a marked reduction of GFP+ cells is observed over time compared with the control and the other shRNAs cells (FIG. 6). This is indicative of a negative effect of shRNA L9 and G5 on normal cell function. SupT1 cells were transduced, but not sorted. The % GFP positivity was measured over a two week time period after transduction (FIG. 7). G5 again shows a reduction of GFP+ cells over time. Thus, both indicate that there is a negative impact on the cell function when expressing shRNA G5.

### Discussion

Comparing cell growth of treated and non-treated cells in an in vitro co-culture system provides a sensitive means to score adverse effects of shRNAs on normal cell function. No adverse effects were observed for the control vector or shRNAs L4, P1, P47 and RT5. However, for the shRNA G5 and L9, cell growth was reduced compared with the control and other shRNA cells. Early detection of cell growth problems induced by shRNAs prevents the extensive testing of shRNAs that will cause adverse side effects.

TABLE 1

| | |
|---|---|
| L2 | AGGAGAGAGAUGGGUGCGA |
| L3 | GGAGAGAGAUGGGUGCGAG |
| L4 | GAGAGAGAUGGGUGCGAGA |
| L5 | AGAGAGAUGGGUGCGAGAG |
| L8 | GAGAUGGGUGCGAGAGCGU |

TABLE 2

| | |
|---|---|
| P44 | AAGGUGAAGGGGCAGUAGU |
| P45 | AGGUGAAGGGGCAGUAGUA |
| P47 | GUGAAGGGGCAGUAGUAAU |

### REFERENCES

Anderson et al, 2003. Oligonucleotides 13:303-312
Das et al, 2004. Journal of Virology, Vol 78, No. 5 pages 2601-2605
Konstantinova, P., P. de Haan, A. T. Das, and B. Berkhout. 2006. Hairpin-induced tRNA-mediated (HITME) recombination in HIV-1. Nucleic Acids Res. 34:2206-2218
Ter Brake et al, 2006. Mol. Ther. Vol 14, No. 6, 883-892
Ter Brake et al, 2008. Mol. Ther, Vol 16, No. 3, 557-564
Ter Brake, O., N. Legrand, K. J. von Eije, M. Centlivre, H. Spits, K. Weijer, B. Blom, and B. Berkhout. 2009. Evaluation of safety and efficacy of RNAi against HIV-1 in the human immune system (Rag-2(−/−)(c)(−/−)) mouse model. Gene Ther. 16(1): 148-53

1-21. (canceled)

22. A compound, composition or kit of parts comprising one or more nucleic acid molecule(s), comprising at least three nucleic acid sequences, each nucleic acid sequence having a length of at least 15 nucleotides, said at least three nucleic acid sequences being selected from the group consisting of a nucleic acid sequence which is at least 75% complementary to a sequence of Table 1 (L2, L3, L4, L5 or L8) and a nucleic acid sequence which is at least 70% complementary to a sequence of Table 2 (P47, P44 or P45) and a nucleic acid sequence which is at least 70% complementary to the sequence ACAGGAGCAGAUGAUACAG (P1) and a nucleic acid sequence which is at least 70% complementary to the sequence AUGGCAGGAAGAAGCGGAG (RT5), wherein said one or more nucleic acid molecule(s) do not comprise a nucleic acid sequence which is complementary to the sequence GAAGAAAUGAUGACAGCAU (G5).

23. A compound, composition or kit of parts according to claim 22, with the proviso that, if said nucleic acid molecule(s) comprise a nucleic acid sequence which is at least 75% complementary to a sequence of Table 1, then said nucleic acid molecule(s) comprise at least two other nucleic acid sequences which are not at least 75% complementary to a sequence of Table 1 and with the proviso that, if said nucleic acid molecule(s) comprise a nucleic acid sequence which is at least 70% complementary to a sequence of Table 2, then said nucleic acid molecule(s) comprise at least two other nucleic acid sequences which are not at least 70% complementary to a sequence of Table 2.

24. A compound, composition or kit of parts according to claim 22, wherein said one or more nucleic acid molecule(s) comprise at least three nucleic acid sequences being selected from the group consisting of a nucleic acid sequence which is at least 75% complementary to the sequence GAGAGAGAUGGGUGCGAGA (L4) and a nucleic acid sequence which is at least 70% complementary to the sequence ACAGGAGCAGAUGAUACAG (P1) and a nucleic acid sequence which is at least 70% complementary to the sequence GUGAAGGGGCAGUAGUAAU (P47) and a nucleic acid sequence which is at least 70% complementary to the sequence AUGGCAGGAAGAAGCGGAG (RT5).

25. A pharmaceutical composition comprising a compound, composition or kit of parts according to claim 22 or 38 and a pharmaceutically acceptable diluent, carrier or excipient.

26. A gene delivery vehicle or an isolated or recombinant host cell comprising one or more nucleic acid molecule(s) according to claim 22 or 38.

27. A method for at least in part inhibiting replication and/or proliferation of HIV, comprising contacting said HIV with a compound or composition or kit of parts according to claim 22 or 25 or 26 or 38.

28. A method for at least in part treating or preventing AIDS, comprising administering to a subject suffering, or at risk of suffering, from AIDS a therapeutically active amount or a prophylactically active amount of a compound, or a composition, or a kit of parts, or a pharmaceutical composition, or a gene delivery vehicle according to claim 22 or 25 or 26 or 38.

29. A method for determining whether a candidate compound which is capable of inhibiting a pathogenic micro-organism is suitable for development of a medicament, the method comprising:
introducing said candidate compound into a cell wherein said pathogenic micro-organism, if present, is capable of replicating and/or proliferating and determining whether said cell comprising said candidate compound exhibits between 85% and 115%, preferably between 90% and 110%, most preferably between 95% and 105% cell function as compared to the same kind of cell which has not been provided with said candidate compound, which indicates that said candidate compound is suitable for development of a medicament, and
introducing said candidate compound into a cell wherein said pathogenic micro-organism, if present, is capable of replicating and/or proliferating, and infecting said cell with said pathogenic micro-organism, at a dose which results in replication of said pathogenic micro-organism in cells which do not comprise said candidate compound, and determining whether replication and/or proliferation of said pathogenic micro-organism in cells comprising said candidate compound is inhibited during culturing for at least 80 days, which indicates that said candidate compound is suitable for development of a medicament; and/or
introducing said candidate compound into a cell wherein said pathogenic micro-organism, if present, is capable of replicating and/or proliferating, and infecting said cell with said pathogenic micro-organism at a dose which firstly results in inhibition of said pathogenic micro-organism during at least 20 days and subsequently results in replication of said pathogenic micro-organism in said cell, and determining whether at least 80% of the replicating micro-organisms are escape mutants of said pathogenic micro-organisms, which indicates that said candidate compound is suitable for development of a medicament.

30. A method according to claim 29, wherein cell function is determined of a cell which comprises said candidate compound and which does not comprise said pathogenic micro-organism.

31. A method according to claim 29, comprising comparing growth and/or proliferation of said cell comprising said candidate compound as compared to the growth and/or proliferation of the same kind of cells which have not been provided with said candidate compound, and determining whether said cell comprising said candidate compound is capable of growing and/or proliferating at a rate of between 85% and 115%, preferably between 90% and 110%, most preferably between 95% and 105% as compared to the same kind of cells which have not been provided with said candidate compound;
wherein, if said cell comprising said candidate compound is capable of growing and/or proliferating at a rate of between 85% and 115%, preferably between 90% and 110%, most preferably between 95% and 105% as compared to the same kind of cells which have not been provided with said candidate compound, said candidate compound is selected as suitable for further testing and/or development of a medicament, and
if said cell comprising said candidate compound is capable of growing and/or proliferating at a rate of less than 85% or more than 115%, as compared to the same kind of cells which have not been provided with said candidate compound, said candidate compound is excluded from development of a medicament.

32. A method according to claim 29, comprising infecting a culture of said cells with said pathogenic micro-organism at a dose of less than 2 ng CA-p24 and determining whether replication and/or proliferation of said pathogenic micro-organism in cells comprising said candidate compound is inhibited during culturing for at least 80 days, preferably at least 100 days.

33. A method according to claim 29, comprising infecting a culture of said cells with said pathogenic micro-organism at a dose of at least 2 ng CA-p24 and determining whether at least 80% of the replicating micro-organisms are escape mutants of said pathogenic micro-organisms.

34. A method according to claim 32 or 33, further comprising selecting or isolating a compound which is capable of inhibiting replication and/or proliferation of said pathogenic micro-organism during culturing for at least 80 days, preferably at least 100 days, and/or selecting or isolating a compound which involves the presence of at least 80% escape mutants of said pathogenic micro-organisms.

35. A method for selecting at least two compounds from a plurality of candidate compounds comprising at least two nucleic acid sequences which are complementary to a genomic sequence of a pathogenic micro-organism and which are capable of inhibiting a pathogenic micro-organism, the method comprising subjecting said plurality of candidate compounds to a method according to claim 29, and selecting from said plurality of candidate compounds at least two, preferably at least three compounds which are suitable for further testing and/or development of a medicament.

36. A method, a compound, a composition, a kit of parts, a use, a pharmaceutical composition, a vector, a gene delivery vehicle or a host cell according to claim 22 or 25 or 26 or 38, wherein said at least one nucleic acid sequence or said combination of nucleic acid sequences comprises at least one short hairpin RNA (shRNA) and/or at least one small interfering RNA (siRNA) and/or at least one microRNA (miRNA) structure.

37. An isolated or recombinant T-cell and/or hematopoietic stem cell and/or hematopoietic progenitor cell comprising one or more nucleic acid molecule(s) according to claim 22 or 38.

38. A composition comprising at least three nucleic acid sequences, each nucleic acid sequence having a length of at least 15 nucleotides, said at least three nucleic acid sequences being selected from the group consisting of a nucleic acid sequence which is at least 75% complementary to a sequence of Table 1 (L2, L3, L4, L5 or L8) and a nucleic acid sequence which is at least 70% complementary to a sequence of Table 2 (P47, P44 or P45) and a nucleic acid sequence which is at least 70% complementary to the sequence ACAGGAGCAGAUGAUACAG (P1) and a nucleic acid sequence which is at least 70% complementary to the sequence AUGGCAGGAAGAAGCGGAG (RT5), wherein said composition does not comprise a nucleic acid sequence which is complementary to the sequence GAAGAAAUGAUGACAGCAU (G5), and wherein said at least three nucleic acid sequences are independently from each other present on one or more nucleic acid molecule(s).
